(19) United States
(12) Patent Application Publication
(10) Pub. No.: US 2025/0221294 A1
(43) Pub. Date: Jul. 3, 2025

(54) POLYMER, ORGANIC SEMICONDUCTOR MATERIAL, STRETCHABLE POLYMER THIN FILM, AND ELECTRONIC DEVICE

(71) Applicants: Samsung Electronics Co., Ltd., Suwon-si (KR); SEOUL NATIONAL UNIVERSITY R&DB FOUNDATION, Seoul (KR)

(72) Inventors: Hyung Jun KIM, Suwon-si (KR); Yeongkwon KANG, Seoul (KR); Min Sang KWON, Seoul (KR); Hyun Bum KANG, Suwon-si (KR); Gae Hwang LEE, Suwon-si (KR)

(73) Assignees: Samsung Electronics Co., Ltd., Suwon-si (KR); SEOUL NATIONAL UNIVERSITY R&DB FOUNDATION, Seoul (KR)

(21) Appl. No.: 19/006,820

(22) Filed: Dec. 31, 2024

(30) Foreign Application Priority Data
Jan. 2, 2024 (KR) 10-2024-0000562

(51) Int. Cl. H10K 85/10; C08G 61/12; H10K 10/46

(52) U.S. Cl. CPC H10K 85/113; C08G 61/126; H10K 85/151; C08G 2261/124; C08G 2261/1412; C08G 2261/3223; C08G 2261/3225; C08G 2261/3226; C08G 2261/3241; C08G 2261/3243; C08G 2261/414; C08G 2261/51; C08G 2261/92; H10K 10/488

(57) ABSTRACT

Disclosed are a polymer including a structural unit represented by Chemical Formula 1, an organic semiconductor material, and a stretchable polymer thin film and an electronic device including the same.

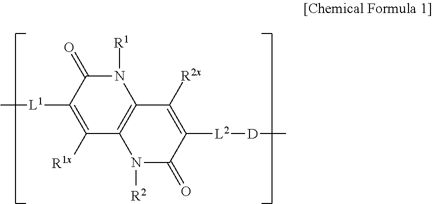

The definition of Chemical Formula 1 is as described in the detailed description.

### CROSS-REFERENCE TO RELATED APPLICATION

This application claims priority to and the benefit of Korean Patent Application No. 10-2024-0000562 filed in the Korean Intellectual Property Office on Jan. 2, 2024, the entire contents of which are incorporated herein by reference.

### BACKGROUND

### 1. Field

Example embodiments relate to polymers, organic semiconductor materials, stretchable polymer thin films, and electronic devices.

### 2. Description of the Related Art

In recent years, research on a stretchable display device and/or a bio-attachable device such as a smart skin device, a soft robot, and a biomedical device has been conducted. In addition to being comprised of materials with suitable electrical properties, these devices should have stretchability according to external movements, and at the same time should be able to maintain their original performance after being restored, and thus research for a new material suitable for this is being conducted.

### SUMMARY

Some example embodiments provide a polymer having relatively excellent electrical properties (such as charge mobility and stretchability).

Some example embodiments provide an organic semiconductor material including the polymer.

Some example embodiments provide a stretchable polymer thin film including the polymer or organic semiconductor material.

Some example embodiments provide an electronic device including the polymer, the organic semiconductor material, or the stretchable polymer thin film.

According to some example embodiments, a polymer including a structural unit represented by Chemical Formula 1 is provided.

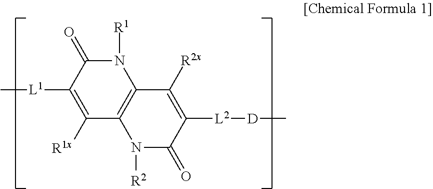

In Chemical Formula 1,
R¹ and R² may each independently be a substituted or unsubstituted C1 to C50 alkyl group, a substituted or unsubstituted C1 to C50 alkoxy group, a substituted or unsubstituted C6 to C50 aryl group, a substituted or unsubstituted C2 to C50 heteroaryl group, a substituted or unsubstituted C3 to C50 cycloalkyl group, a substituted or unsubstituted C3 to C50 heterocycloalkyl group, a substituted or unsubstituted C7 to C50 aralkyl group, —COR^{a}, —OC(═O)R^{b}, —C(═O)OR^{c}, —OC(═O)OR^{d}, —NR^{e}R^{f}, a cyano group, or a combination thereof,
R^{a} to R^{f} are each independently hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a cyano group, or a combination thereof,
R^{1x} and R^{2x} may each independently be hydrogen, deuterium, or a C1 to C10 alkyl group,
L¹ and L² may each independently be a substituted or unsubstituted Se-containing aromatic ring, a substituted or unsubstituted Te-containing aromatic ring, or a fused ring thereof, and
D may be a substituted or unsubstituted C6 to C30 arylene group; a substituted or unsubstituted divalent C3 to C30 heterocyclic group including at least one selected from N, O, S, Se, Te, and Si; a fused ring thereof; or a combination thereof.

In some example embodiments, in Chemical Formula 1, D may be at least one substituted or unsubstituted phenylene group; at least one substituted or unsubstituted naphthylene group; at least one substituted or unsubstituted anthracenylene group; at least one substituted or unsubstituted phenanthrenylene group; at least one substituted or unsubstituted pentagonal rings including at least one of N, O, S, Se, Te, or Si; a fused ring of two or more of the substituted or unsubstituted pentagonal rings; a fused ring of at least one of the substituted or unsubstituted pentagonal rings and at least one substituted or unsubstituted phenylene group; a fused ring of at least one substituted or unsubstituted pentagonal ring and at least one substituted or unsubstituted naphthylene group; a fused ring of at least one substituted or unsubstituted pentagonal ring and at least one substituted or unsubstituted anthracenylene group a fused ring of at least one substituted or unsubstituted pentagonal ring and at least one substituted or unsubstituted phenanthrenylene group; or a combination thereof.

In Chemical Formula 1, D may be, for example, one of an electron donating moiety listed in Group 1.

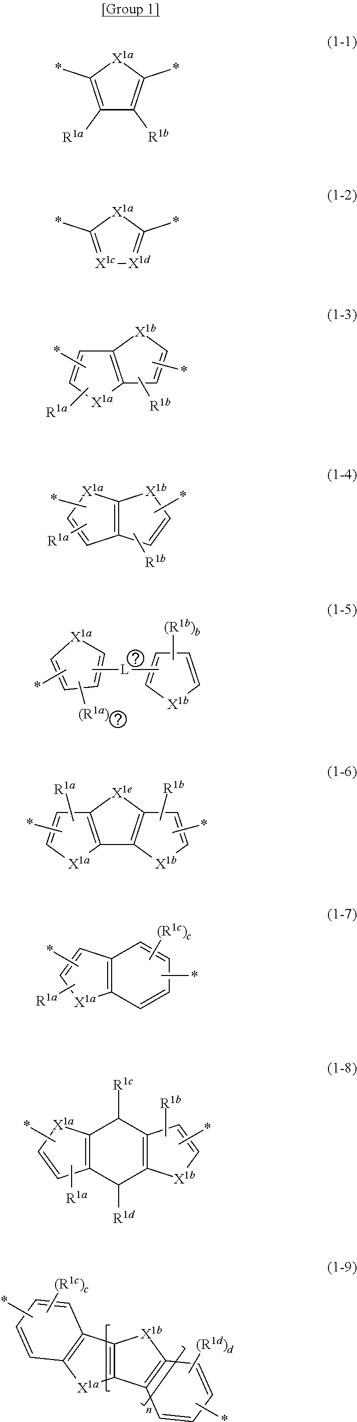

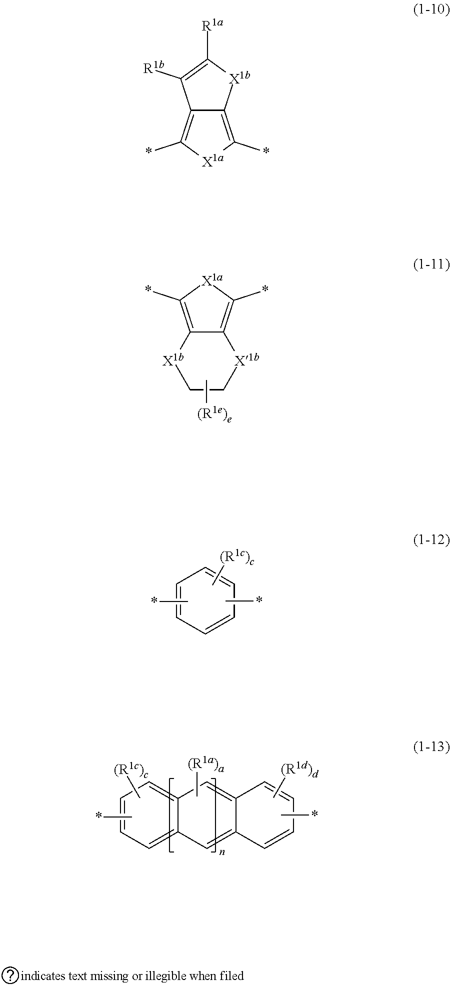

In Group 1,
X^{1a}, X^{1b}, and X′^{1b} may each independently be O, S, Se, or Te,
X^{1c} and X^{1d} may each independently be N, CR^{x}, or SiR^{y},
X^{1e} may be O, S, Se, Te, NR^{v}, CR^{w}R^{x}, or SiR^{y}R^{z},
R^{1a}, R^{1b}, R^{1c}, R^{1d}, R^{1e}, R^{x}, R^{y}, R^{v}, R^{w}, and R^{z} may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C3 to C30 heteroaryl group, a substituted or unsubstituted C7 to C30 alkylaryl group, a substituted or unsubstituted C6 to C30 aryloxy group, a substituted or unsubstituted C3 to C30 heterocyclic group, a halogen, a cyano group, or a combination thereof,
L^{a} may be a single bond or a substituted or unsubstituted C2 to C10 alkenylene group,
a and b may each independently represent an integer from 1 to 2,
c and d may each independently represent an integer from 1 to 3,
e may represent an integer from 1 to 4,
n may represent 0, 1 or 2, and
* may represent a linking point with a main chain of Chemical Formula 1.

In Chemical Formula 1, L¹ and L² may each independently be independently selected from Group 2.

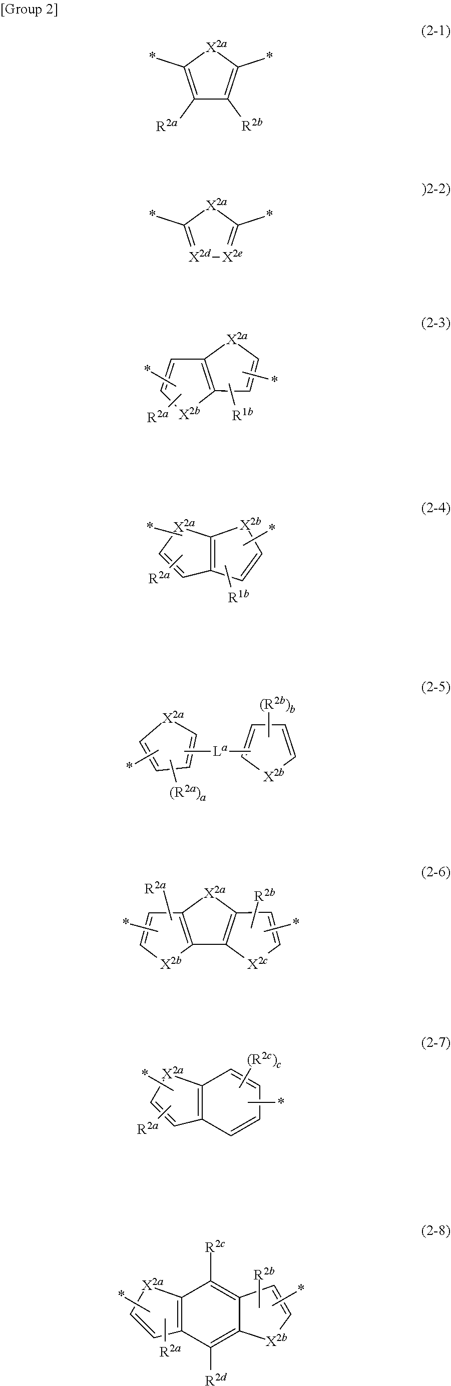

In Group 2,
X^{2a} may be Se or Te,
X^{2b} and X^{2c} may each independently be O, S, Se, or Te,
X^{2d} and X^{2e} may each independently be N, CR^{x} or SiR^{y},
R^{2a}, R^{2b}, R^{2c}, R^{2d}, R^{x}, and R^{y} may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C3 to C30 heteroaryl group, substituted or unsubstituted C7 to C30 alkylaryl group, a substituted or unsubstituted C6 to C30 aryloxy group, a substituted or unsubstituted C3 to C30 heterocyclic group, a cyano group, or a combination thereof,
L^{a} may be a single bond or a substituted or unsubstituted C2 to C10 alkenylene group,
a and b may each independently represent an integer from 1 to 2,
c may represent an integer from 1 to 3,
c may represent an integer from 1 to 3, and
* may represent a linking point with a main chain of Chemical Formula 1.

In Chemical Formula 1, L¹ and L² may each independently be a substituted or unsubstituted selenophene, a substituted or unsubstituted tellurophene, or a fused ring thereof.

In Chemical Formula 1, R¹ and R¹ may each independently be a substituted or unsubstituted C6 to C30 linear alkyl group or a substituted or unsubstituted C6 to C30 branched alkyl group.

In the polymer, an aspect ratio (Z/X) obtained by dividing a length of the shortest axis (Z) of the structural unit of Chemical Formula 1 by a length of the longest axis (X) may be less than or equal to about 0.407.

According to some example embodiments, an organic semiconductor material including the polymer is provided.

According to some example embodiments, a stretchable polymer thin film including the polymer or the organic semiconductor material is provided.

The stretchable polymer thin film may further include an elastomer.

The stretchable polymer thin film may have a charge mobility change of less than or equal to about 10% when stretched by about 30%.

According to some example embodiments, a thin film transistor includes a gate electrode, an organic semiconductor overlapped with the gate electrode, and a source electrode and a drain electrode electrically connected to the semiconductor, wherein the organic semiconductor includes the aforementioned polymer.

According to some example embodiments, an electronic device including the aforementioned polymer is provided.

According to some example embodiments, an electronic device including the stretchable polymer thin film is provided.

The electronic device may include an organic photoelectric device, an organic light emitting device, an organic diode, an organic thin film transistor, an organic solar cell, or an attachable device.

The polymer may satisfy electrical characteristics (such as charge mobility, and stretchability simultaneously), and thus may be effectively applied to stretchable electronic devices.

### BRIEF DESCRIPTION OF THE DRAWINGS

FIGS. 1 to 3 are cross-sectional views each showing a thin film transistor according to some example embodiments.

### DETAILED DESCRIPTION

Hereinafter, embodiments are described in detail so that those skilled in the art can easily implement them. However, the actual applied structure may be implemented in various different forms and is not limited to the implementations described herein.

In the drawings, the thickness of layers, films, panels, regions, etc., are exaggerated for clarity.

It will be understood that when an element such as a layer, film, region, or substrate is referred to as being “on” another element, it may be directly on the other element or intervening elements may also be present. In contrast, when an element is referred to as being “directly on” another element, there are no intervening elements present.

As used herein, “at least one of A, B, or C,” “one of A, B, C, or a combination thereof” and “one of A, B, C, and a combination thereof” refer to each constituent element, and a combination thereof (e.g., A; B; C; A and B; A and C; B and C; or A, B, and C).

Hereinafter, the terms “lower” and “upper” are used for better understanding and ease of description, but do not limit the location relationship.

It will further be understood that when an element is referred to as being “on” another element, it may be above or beneath or adjacent (e.g., horizontally adjacent) to the other element. It will also be understood that spatially relative terms, such as “above”, “top”, etc., are intended to encompass different orientations of the device in use or operation in addition to the orientation depicted in the figures, and that the device may be otherwise oriented (rotated 90 degrees or at other orientations) and the spatially relative terms used herein interpreted accordingly. It will be understood that elements and/or properties thereof (e.g., structures, surfaces, directions, or the like), which may be referred to as being “perpendicular,” “parallel,” “coplanar,” or the like with regard to other elements and/or properties thereof (e.g., structures, surfaces, directions, or the like) may be “perpendicular,” “parallel,” “coplanar,” or the like or may be “substantially perpendicular,” “substantially parallel,” “substantially coplanar,” respectively, with regard to the other elements and/or properties thereof. Elements and/or properties thereof (e.g., structures, surfaces, directions, or the like) that are “substantially perpendicular” with regard to other elements and/or properties thereof will be understood to be “perpendicular” with regard to the other elements and/or properties thereof within manufacturing tolerances and/or material tolerances and/or have a deviation in magnitude and/or angle from “perpendicular,” or the like with regard to the other elements and/or properties thereof that is equal to or less than 10% (e.g., a. tolerance of ±10%). Elements and/or properties thereof (e.g., structures, surfaces, directions, or the like) that are “substantially parallel” with regard to other elements and/or properties thereof will be understood to be “parallel” with regard to the other elements and/or properties thereof within manufacturing tolerances and/or material tolerances and/or have a deviation in magnitude and/or angle from “parallel,” or the like with regard to the other elements and/or properties thereof that is equal to or less than 10% (e.g., a. tolerance of ±10%). Elements and/or properties thereof (e.g., structures, surfaces, directions, or the like) that are “substantially coplanar” with regard to other elements and/or properties thereof will be understood to be “coplanar” with regard to the other elements and/or properties thereof within manufacturing tolerances and/or material tolerances and/or have a deviation in magnitude and/or angle from “coplanar,” or the like with regard to the other elements and/or properties thereof that is equal to or less than 10% (e.g., a. tolerance of ±10%). It will be understood that elements and/or properties thereof may be recited herein as being “identical” to, “the same” or “equal” as other elements, and it will be further understood that elements and/or properties thereof recited herein as being “identical” to, “the same” as, or “equal” to other elements may be “identical” to, “the same” as, or “equal” to or “substantially identical” to, “substantially the same” as or “substantially equal” to the other elements and/or properties thereof. Elements and/or properties thereof that are “substantially identical” to, “substantially the same” as or “substantially equal” to other elements and/or properties thereof will be understood to include elements and/or properties thereof that are identical to, the same as, or equal to the other elements and/or properties thereof within manufacturing tolerances and/or material tolerances. Elements and/or properties thereof that are identical or substantially identical to and/or the same or substantially the same as other elements and/or properties thereof may be structurally the same or substantially the same, functionally the same or substantially the same, and/or compositionally the same or substantially the same. It will be understood that elements and/or properties thereof described herein as being the “substantially” the same and/or identical encompasses elements and/or properties thereof that have a relative difference in magnitude that is equal to or less than 10%. Further, regardless of whether elements and/or properties thereof are modified as “substantially,” it will be understood that these elements and/or properties thereof should be construed as including a manufacturing or operational tolerance (e.g., ±10%) around the stated elements and/or properties thereof. When the terms “about” or “substantially” are used in this specification in connection with a numerical value, it is intended that the associated numerical value include a tolerance of ±10% around the stated numerical value. When ranges are specified, the range includes all values therebetween such as increments of 0.1%.

As used herein, when a definition is not otherwise provided, “combination thereof” in the definition of chemical formula refers to at least two substituents bound to each other by a single bond or a C1 to C10 alkylene group, or at least two fused substituents.

In addition, hereinafter, when a definition is not otherwise provided, “combination” may refer to a mixture of two or more, an alloy of two or more, and a stacked structure of two or more.

As used herein, when a definition is not otherwise provided, “substituted” refers to replacement of hydrogen of a compound or a moiety by a substituent selected from a hydroxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a C1 to C30 alkyl group, a C2 to C30 alkenyl group, a C2 to C30 alkynyl group, a C6 to C30 aryl group, a C3 to C30 heteroaryl group, a C7 to C30 arylalkyl group, a C1 to C30 alkoxy group, a C1 to C20 heteroalkyl group, a C3 to C20 heterocyclic group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, or a combination thereof.

As used herein, when a definition is not otherwise provided, “hetero” refers to one including 1 to 4 heteroatoms selected from N, O, S, Se, Te, Si and P.

As used herein, when a definition is not otherwise provided, “alkyl group” may be a C1 to C30 (e.g., C1 to C20) linear or branched, saturated, monovalent hydrocarbon group (e.g., a methyl group, an ethyl group, a propyl group, an isobutyl group, a sec-butyl group, a tert-butyl group, a pentyl group, an iso-amyl group, a hexyl group, and the like).

As used herein, when a definition is not otherwise provided, “alkoxy group” may refer to a C1 to C30 (e.g., C1 to C20) alkyl group that is linked via an oxygen, e.g., a methoxy group, an ethoxy group, and a sec-butoxy group.

As used herein, when a definition is not otherwise provided, “aryl group” refers to a monovalent functional group formed by the removal of one hydrogen atom from one or more rings of C6 to C30 (e.g., C6 to C20) arene, for example a phenyl group or a naphthyl group. The arene refers to a hydrocarbon having an aromatic ring, and includes monocyclic and polycyclic hydrocarbons wherein the additional ring(s) of the polycyclic hydrocarbon may be aromatic or nonaromatic.

As used herein, when a definition is not otherwise provided, “heteroaryl group” includes at least one heteroatom such as N, O, S, Se, Te, Si, or P in the aforementioned aryl group includes at least one heteroatom such as N, O, S, Se, Te, Si, and P and the remaining carbon.

As used herein, when a definition is not otherwise provided, “aralkyl group” may refer to a group represented by —(CR^{2a}R^{2b})ₘAr, wherein R^{2a} and R^{2b} are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C3 to C30 heteroaryl group, substituted or unsubstituted C7 to C30 alkylaryl group, a substituted or unsubstituted C6 to C30 aryloxy group, a substituted or unsubstituted C3 to C30 heterocyclic group, a halogen, or a cyano group, and Ar is the aforementioned aryl group or heteroaryl group.

As used herein, when a definition is not otherwise provided, “heterocyclic group” includes at least one heteroatom such as N, O, S, Se, Te, Si, or P in a ring such as a C6 to C30 (e.g., C6 to C20) aryl group, a C3 to C30 (e.g., C3 to C20) cycloalkyl group, a fused ring thereof, or a combination thereof, and the remaining carbon. When the heterocyclic group is a fused ring, a heteroatom may be included in the entire heterocyclic group or at least one of the rings.

As used herein, when a definition is not otherwise provided, “aromatic ring” refers to a functional group in which all atoms in the cyclic functional group have a pi-orbital, and wherein these pi-orbitals are conjugated. For example, the aromatic ring may be a (e.g., C6 to C20) C6 to C30 aryl group or C3 to C30 (e.g., C3 to C20) heteroaryl group.

Hereinafter, a device, layer, polymer semiconductor, element, region, or the like that is described as being “stretchable” will be understood to be elastic and/or configured to be elastic, such that the device, layer, polymer semiconductor, element, region, or the like is configured to be elastically deformed (e.g., stretched, compressed, subjected to strain, etc.) such that the device, layer, polymer semiconductor, element, region, or the like is configured to resume its same original shape after being deformed. For example, a stretchable device, layer, polymer semiconductor, element, region, or the like as described herein may be configured to be elastically deformed such that the stretchable device, layer, polymer semiconductor, element, region, or the like can resume, and does resume, an original shape after being stretched or compressed within an operational range. Hereinafter, a device, layer, polymer semiconductor, element, region, or the like that is described as being “non-stretchable” or “rigid” will be understood to be non-elastic and/or not configured to be elastic, such that the device, layer, element, region, or the like is configured to not be elastically deformed (e.g., stretched, compressed, subjected to strain, etc.) such that the device, layer, polymer semiconductor, element, region, or the like is configured to not resume its same original shape after being deformed. For example, a non-stretchable device, layer, polymer semiconductor, element, region, or the like as described herein may not be able to be elastically deformed due to applied strain such that the non-stretchable device, layer, polymer semiconductor, element, region, or the like cannot, and does not, resume an original shape after being stretched or compressed.

Hereinafter, a polymer according to some example embodiments is described.

The polymer according to some example embodiments may be a stretchable polymer, or may include a structural unit represented by Chemical Formula 1.

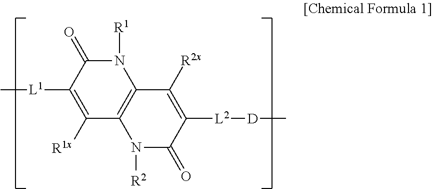

In Chemical Formula 1,
R¹ and R² may each independently be a substituted or unsubstituted C1 to C50 (C1 to C40 or C1 to C30) alkyl group, a substituted or unsubstituted C1 to C50 (C1 to C40 or C1 to C30) alkoxy group, a substituted or unsubstituted C6 to C50 (C6 to C40 or C6 to C30) aryl group, a substituted or unsubstituted C2 to C50 (C2 to C40 or C2 to C30) heteroaryl group, a substituted or unsubstituted C3 to C50 (C3 to C40 or C3 to C30) cycloalkyl group, a substituted or unsubstituted C3 to C50 (C3 to C40 or C3 to C30) heterocycloalkyl group, a substituted or unsubstituted C7 to C50 (C7 to C40 or C7 to C30) aralkyl group, —COR^{a}, —OC(═O)R^{b}, —C(═O)OR^{c}, —OC(═O)OR^{d}, —NR^{e}R^{f}, a cyano group, or a combination thereof, wherein R^{a} to R^{f} may each independently be hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a cyano group, or a combination thereof,
R^{1x} and R^{2x} may each independently be hydrogen, deuterium, or a C1 to C10 alkyl group,
L¹ and L² may each independently be a substituted or unsubstituted Se-containing aromatic ring, a substituted or unsubstituted Te-containing aromatic ring, or a fused ring thereof, and
D may be a substituted or unsubstituted C6 to C30 arylene group; a substituted or unsubstituted divalent C3 to C30 heterocyclic group including at least one selected from N, O, S, Se, Te, and Si; a fused ring thereof; or a combination thereof.

The polymer may be a copolymer including a first structural unit including an electron accepting moiety and a second structural unit including an electron donating moiety.

The first structural unit may include an electron accepting moiety which may be a naphthyridine dione (NTD), and linkers of L¹ and L² at both terminal ends of the electron accepting moiety, and these linkers are a substituted or unsubstituted Se-containing aromatic ring, a substituted or unsubstituted Te-containing aromatic ring, or a fused ring thereof. The carbonyl group (C═O) of the electron accepting moiety and Te or Se of the linker may control the torsion angle of the polymer through intramolecular interaction, thereby increasing planarity and improving charge mobility.

In the structural unit represented by Chemical Formula 1, the first structural unit including an electron accepting moiety may provide improved electrical characteristics and high charge mobility due to relatively high crystallinity to the polymer, and the second structural unit including an electron donating moiety may reduce the crystallinity of the polymer, thereby improving stretchability.

For example, in Chemical Formula 1, R¹ and R² may each be a relatively long linear alkyl group or a bulky branched alkyl group; for example, R¹ and R² may each independently be a substituted or unsubstituted C6 to C30 linear alkyl group or a substituted or unsubstituted C6 to C30 branched alkyl group, for example, a substituted or unsubstituted C8 to C30 linear alkyl group or a substituted or unsubstituted C8 to C30 branched alkyl group, a substituted or unsubstituted C10 to C30 linear alkyl group or a substituted or unsubstituted C10 to C30 branched alkyl group. Accordingly, the polymer can have high solubility in organic solvents.

In Chemical Formula 1, D may be the moiety having an electron donating property, for example, a substituted or unsubstituted C6 to C30 arylene group; a substituted or unsubstituted divalent C3 to C30 heterocyclic group including at least one of N, O, S, Se, Te, or Si; fused rings thereof; or a combination thereof. For example, D may be one or more substituted or unsubstituted phenylene group; one or more substituted or unsubstituted naphthylene group; one or more substituted or unsubstituted anthracenylene group; one or more substituted or unsubstituted phenanthrenylene group; one or more substituted or unsubstituted pentagonal ring including at least one of N, O, S, Se, Te, or Si; a fused ring of two or more of the substituted or unsubstituted pentagonal rings; a fused ring of one or more substituted or unsubstituted pentagonal rings and one or more substituted or unsubstituted phenylene group; a fused ring of one or more substituted or unsubstituted pentagonal ring and one or more substituted or unsubstituted naphthylene group; a fused ring of one or more substituted or unsubstituted pentagonal ring and one or more substituted or unsubstituted anthracenylene group; a fused ring of one or more substituted or unsubstituted pentagonal ring and one or more substituted or unsubstituted phenanthrenylene group; or a combination thereof.

In Chemical Formula 1, D may be, for example, one of the electron donating moieties of Group 1, but is not limited thereto.

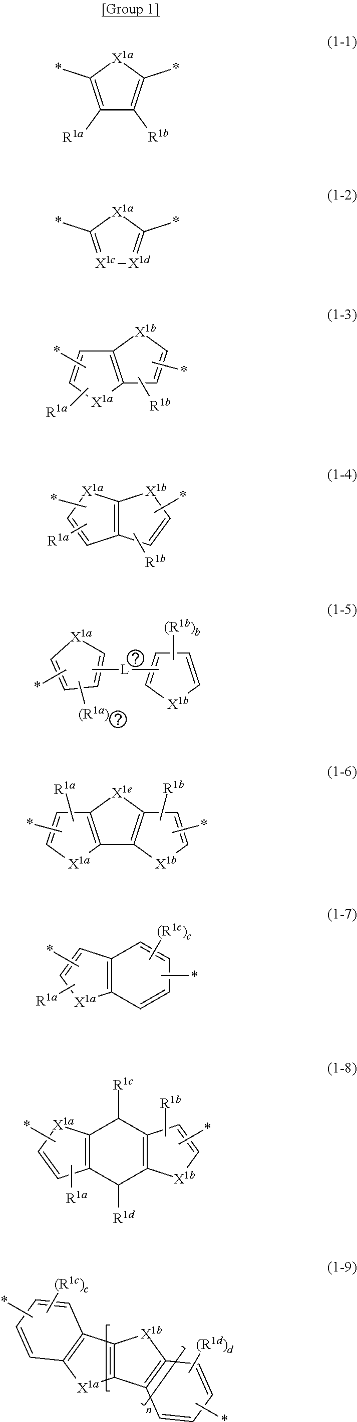

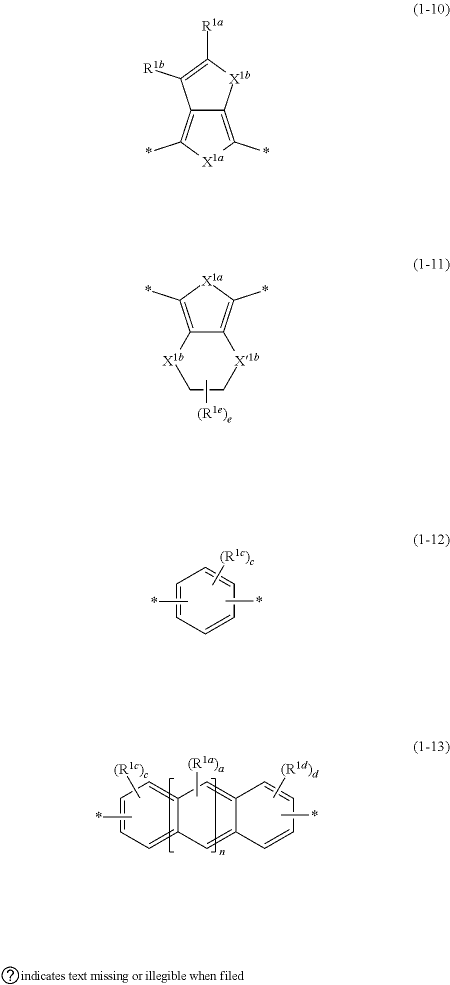

In Group 1,
X^{1a}, X^{1b}, and X′^{1b} may each independently be O, S, Se, or Te,
X^{1c} and X^{1d} may each independently be N, CR^{x}, or SiR^{y},
X^{1e} may be O, S, Se, Te, NR^{v}, CR^{w}R^{x}, or SiR^{y}R^{z},
R^{1a}, R^{1b}, R^{1c}, R^{1d}, R^{1e}, R^{x}, R^{y}, R^{v}, R^{w}, and R^{z} may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C3 to C30 heteroaryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group,
L^{a} may be a single bond or a substituted or unsubstituted C2 to C10 alkenylene group (e.g., vinylene group),
subscripts a and b may each independently be an integer from 1 to 2,
subscripts c and d may each independently be an integer from 1 to 3,
subscript e may be an integer from 1 to 4,
subscript n may be 0, 1, or 2, and
* may be a linking point with a main chain of Chemical Formula 1. For example, * may represent a linking point with L² or L¹.

In Chemical Formula 1, L¹ and L² may each independently be selected from Group 2.

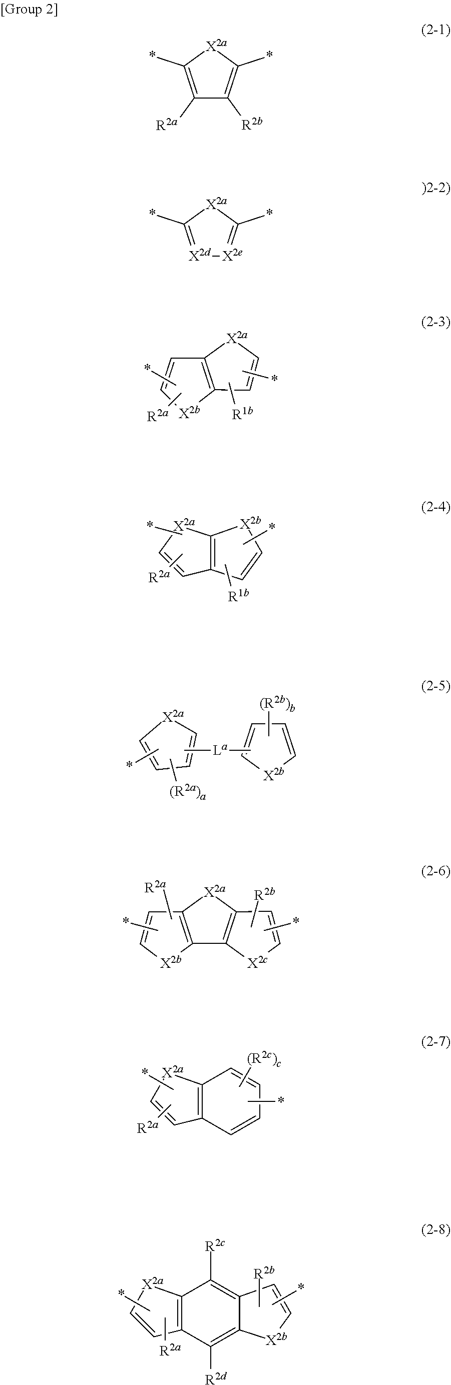

In Group 2,
X^{2a} is Se or Te,
X^{2b} and X^{2c} may each independently be O, S, Se, or Te,
X^{2d} and X^{2e} may each independently be N, CR^{x}, or SiR^{y},
R^{2a}, R^{2b}, R^{2c}, R^{2d}, R^{x}, and R^{y} may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C3 to C30 heteroaryl group, a substituted or unsubstituted C7 to C30 alkylaryl group, a substituted or unsubstituted C6 to C30 aryloxy group, a substituted or unsubstituted C3 to C30 heterocyclic group, or a cyano group,
L^{a} may be a single bond or a substituted or unsubstituted C2 to C10 alkenylene group (e.g., vinylene group),
subscripts a and b may each independently be an integer from 1 to 2,
subscript c may be an integer from 1 to 3, and
* may be a linking point with a main chain of Chemical Formula 1.

In Chemical Formula 1, L¹ and L² may each independently be a substituted or unsubstituted selenophene, a substituted or unsubstituted tellurophene, or a fused ring thereof.

In Chemical Formula 1, R¹ and R² may each independently be a substituted or unsubstituted C6 to C30 linear alkyl group or a substituted or unsubstituted C6 to C30 branched alkyl group.

Examples of the polymer including the structural unit represented by Chemical Formula 1 may be a polymer including a structural unit represented by any one of Groups 3-1 and 3-2.

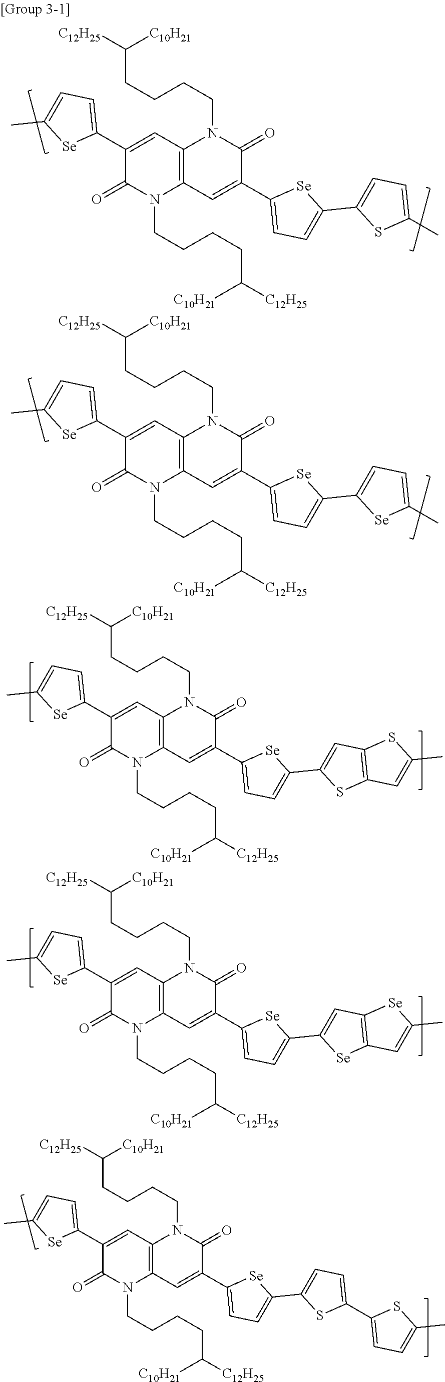

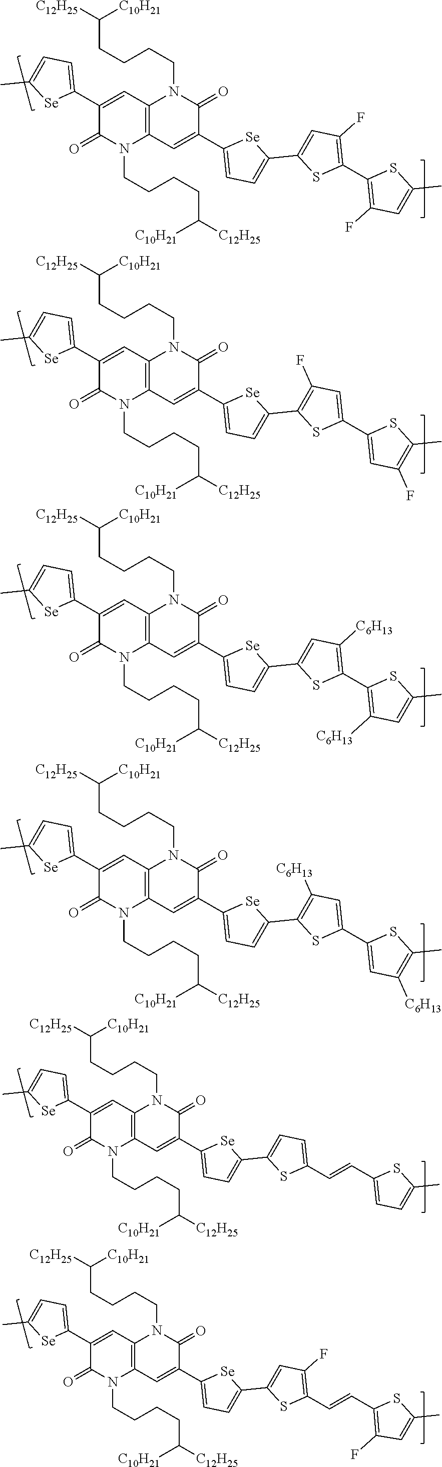

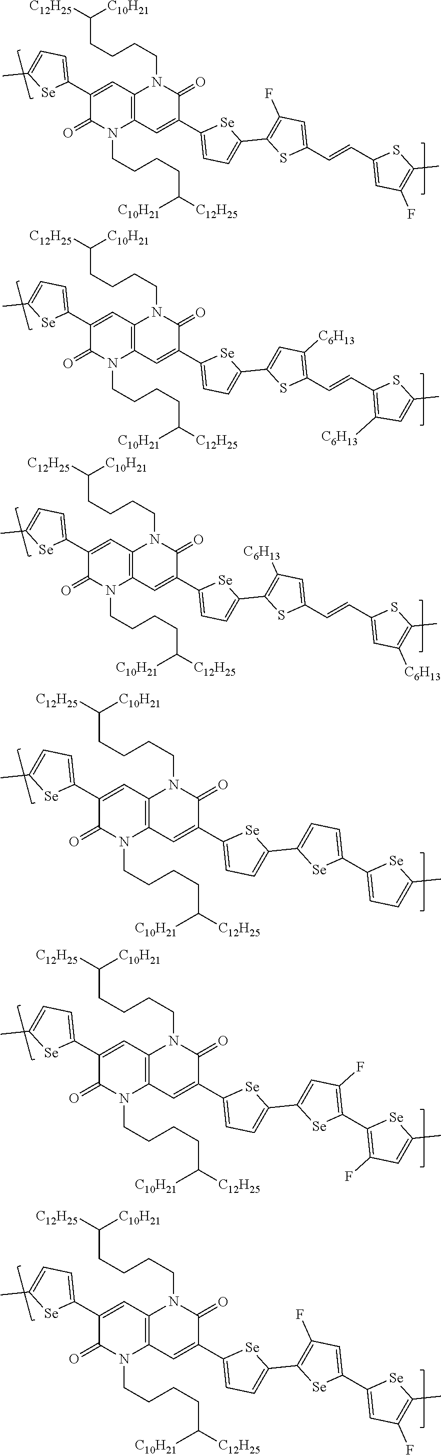

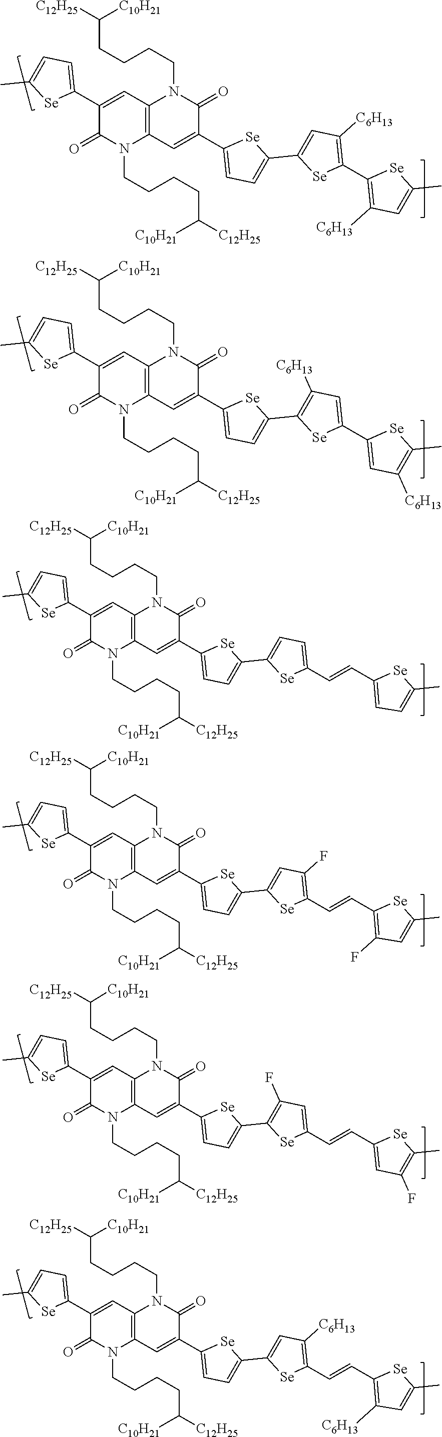

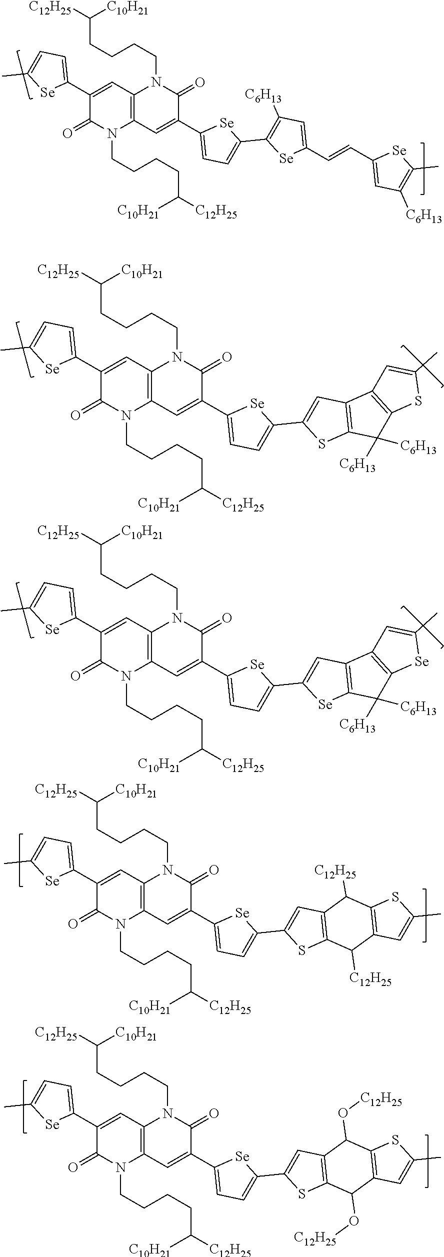

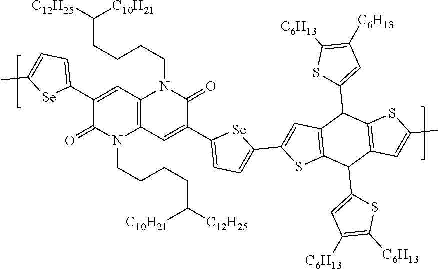

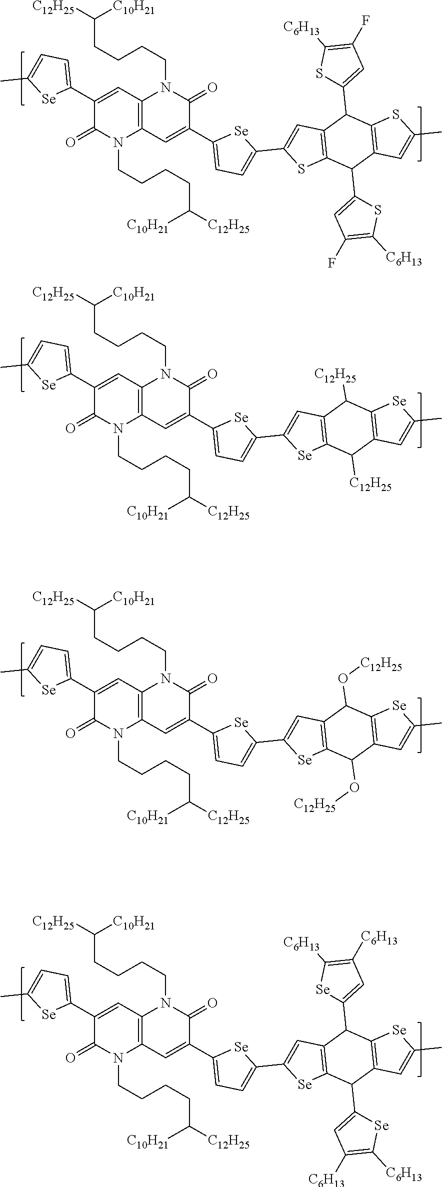

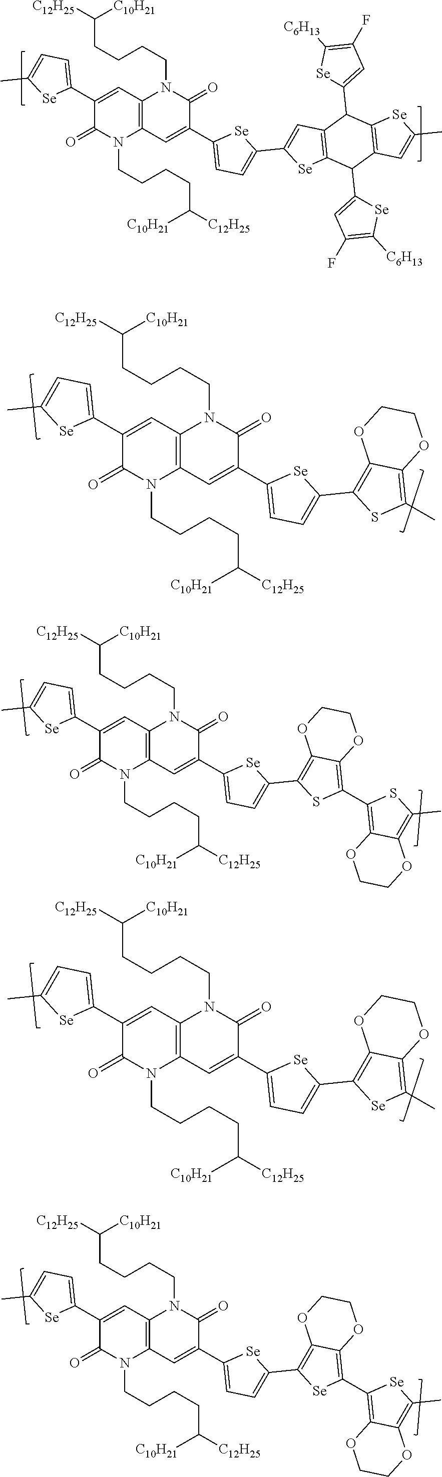

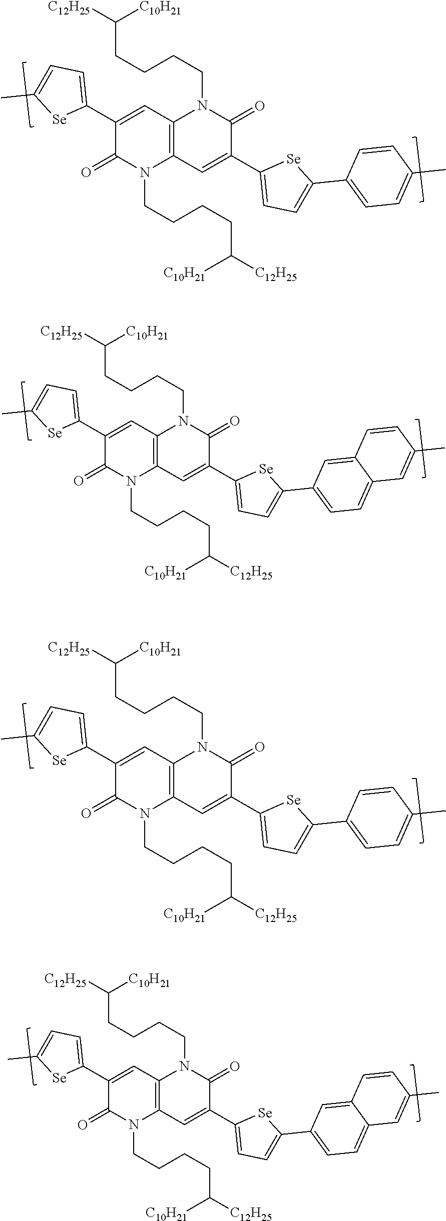

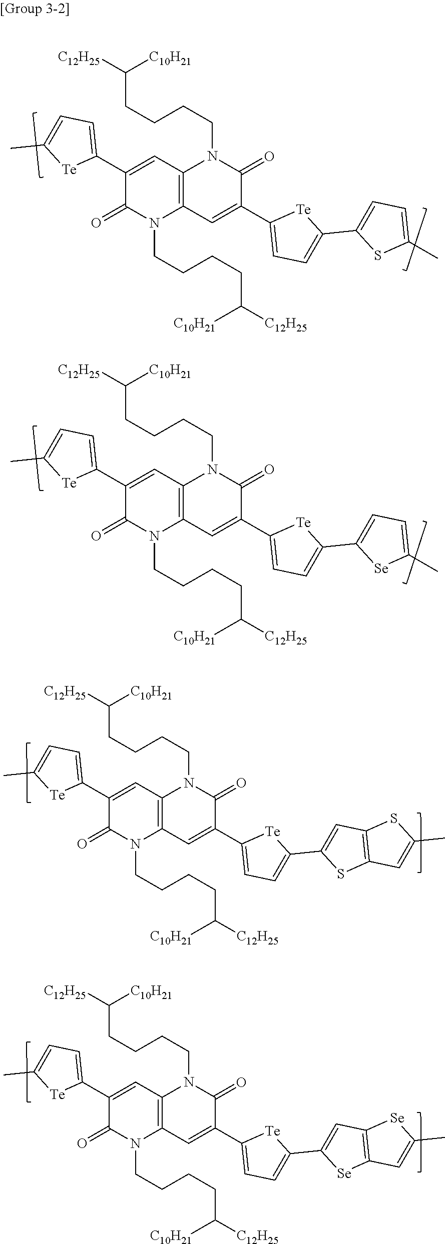

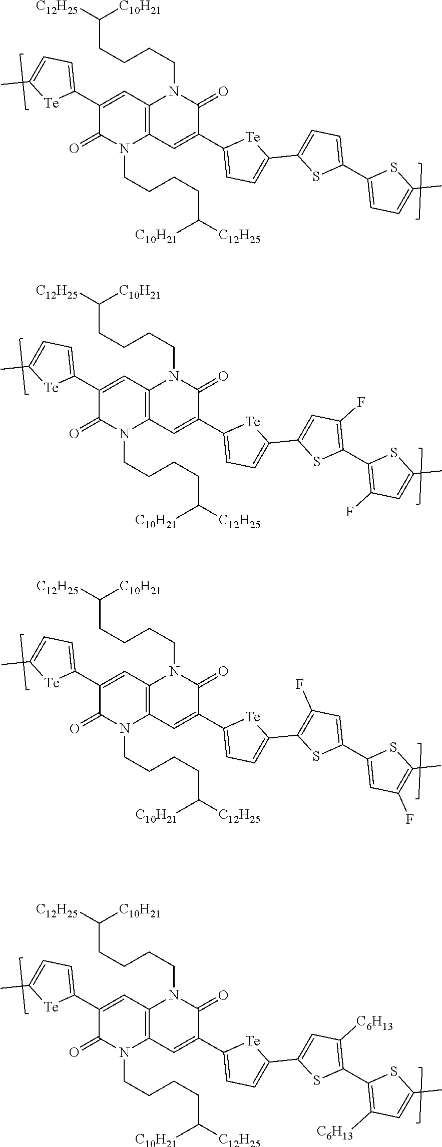

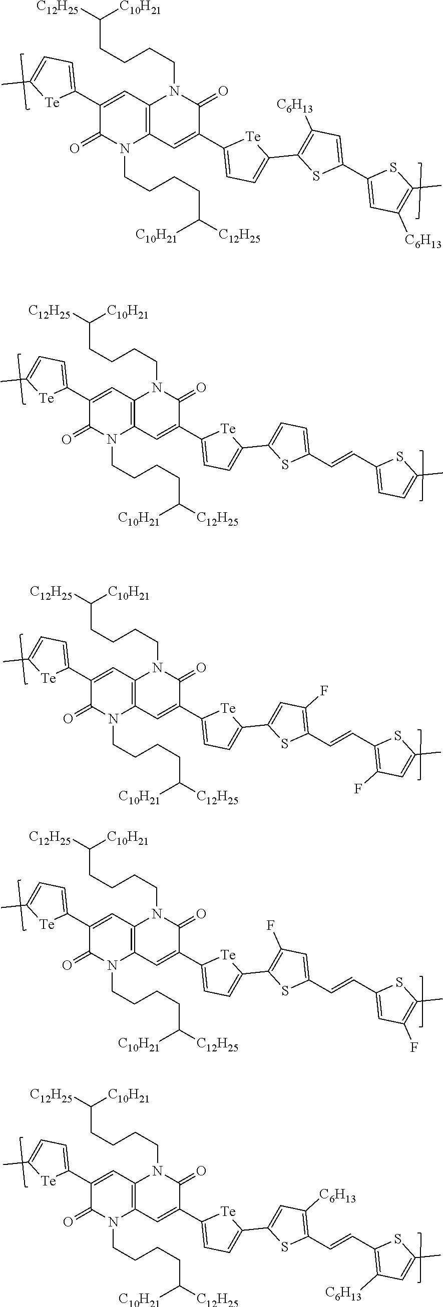

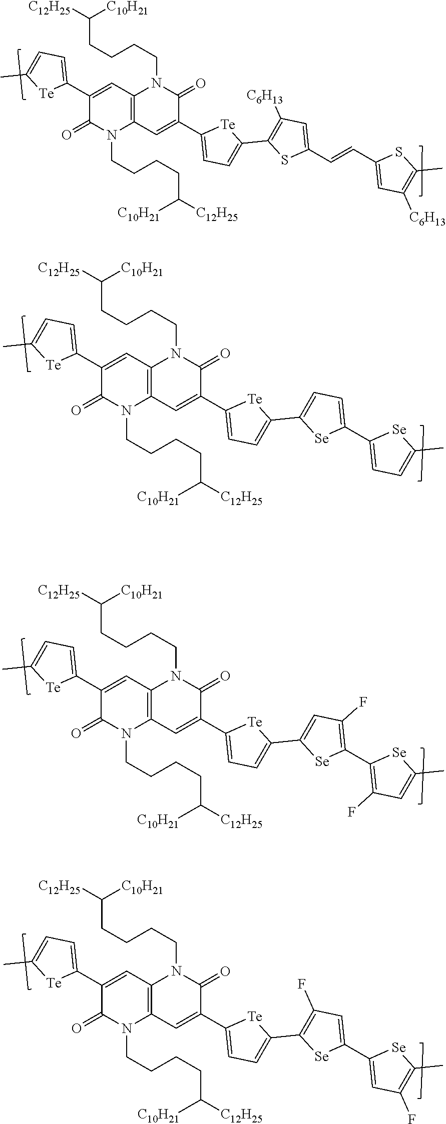

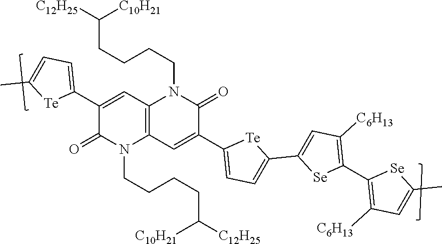

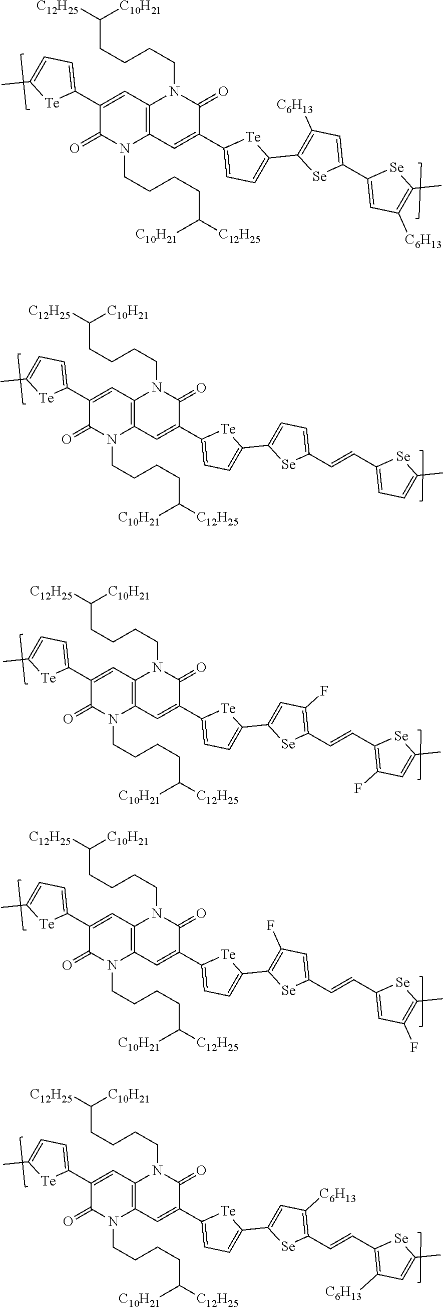

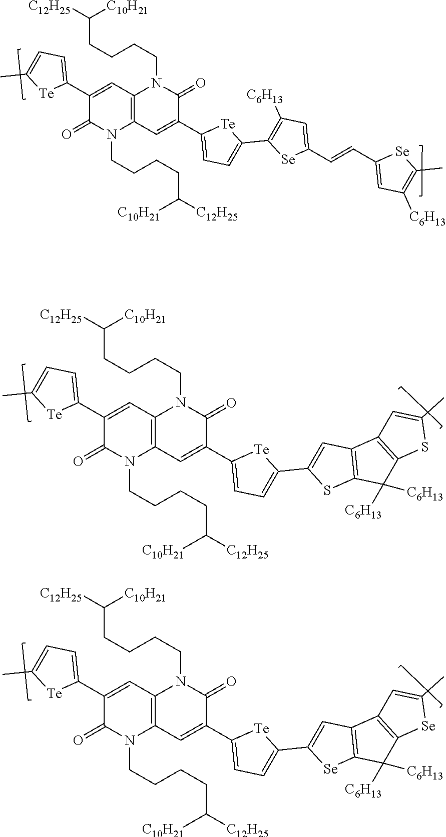

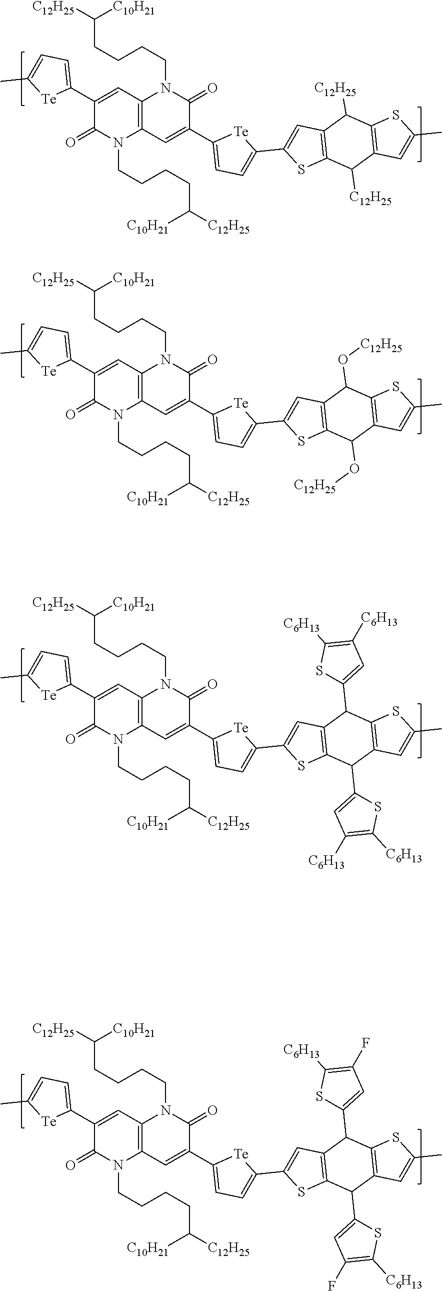

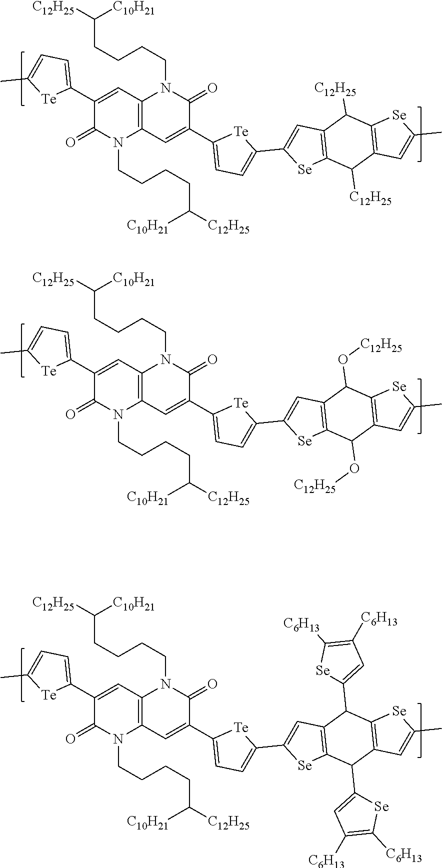

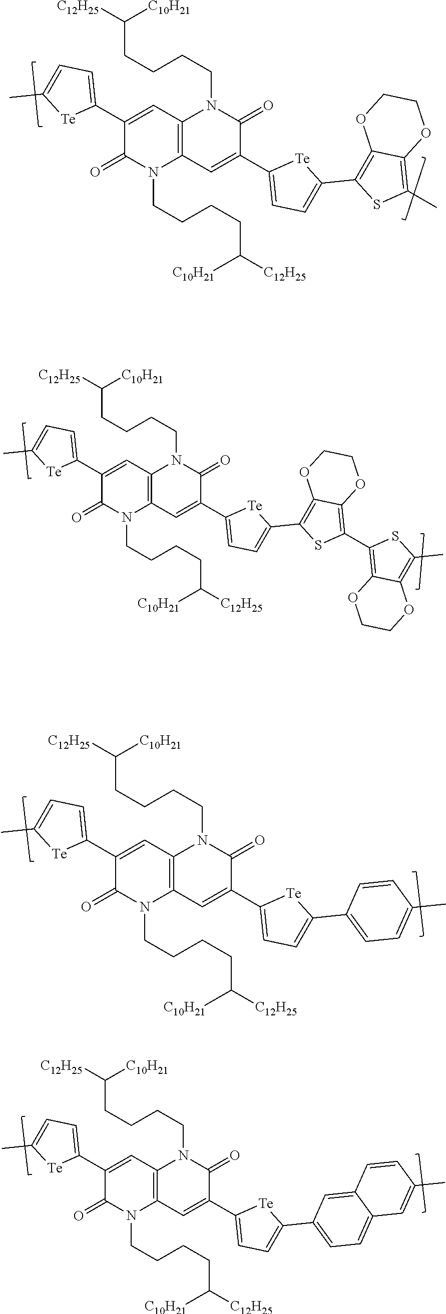

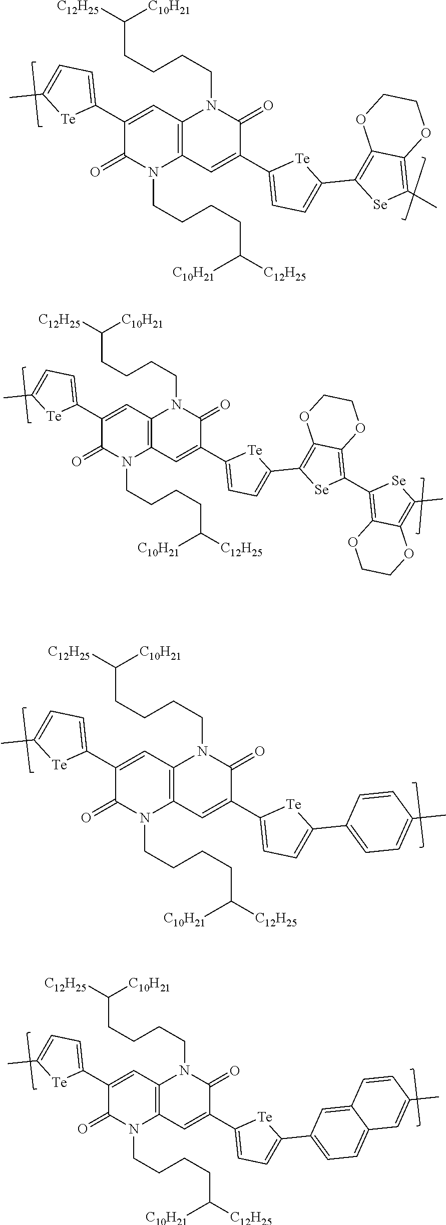

In Groups 3-1 and 3-2, all of the substituents of R¹ and R² of Chemical Formula 1 may be substituted for the 5-decylheptadecyl group substituted on naphthyridine dione.

In addition, in Groups 3-1 and 3-2, —C₆H₁₃ maybe substituted with a linear or branched C1 to C10 alkyl group, and —OC₁₂H₂₅ may be substituted with a linear or branched C1 to C20 alkoxy group.

In addition, the hydrogen of the ring structure of the electron accepting moiety (corresponding to the naphthyridine dione of Chemical Formula 1 and the linker of L¹ and L²) shown in Groups 3-1 and 3-2 may be substituted with a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C3 to C30 heteroaryl group, a substituted or unsubstituted C7 to C30 alkylaryl group, a substituted or unsubstituted C6 to C30 aryloxy group, a substituted or unsubstituted C3 to C30 heterocyclic group, or a cyano group.

In addition, the hydrogen of the ring structure of the electron donating moiety (corresponding to D in Chemical Formula 1) shown in Groups 3-1 and 3-2 may be substituted with a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C3 to C30 heteroaryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group.

The polymer may be, for example, a random copolymer, a block copolymer, or an alternating copolymer.

In the polymer, an aspect ratio (Z/X) obtained by dividing a length of the shortest axis (Z) of the compound composed of one structural unit of Chemical Formula 1 by a length of the longest axis (X) may be less than or equal to about 0.407, or less than or equal to about 0.406. Additionally, the aspect ratio (Z/X) may be greater than or equal to 0.400.

The polymer including a structural unit providing an aspect ratio in the above range may have a relatively high crystallinity and facilitate stacking due to increased interaction between polymer chains. These crystalline regions may accelerate charge flow within a thin film including the polymer, which in turn may improve device mobility.

The number of structural units of Chemical Formula 1 in the polymer may be 3 to 2000, 3 to 1500, 3 to 1000, 3 to 800, 5 to 800, 5 to 700, 5 to 500, or 5 to 300, but is not limited thereto.

In some example embodiments, the structural unit of Chemical Formula 1 may be included in an amount greater than about 50 mol %, for example, greater than or equal to about 55 mol %, greater than or equal to about 60 mol %, greater than or equal to about 65 mol %, greater than or equal to about 70 mol %, greater than or equal to about 75 mol %, or greater than or equal to about 80 mol % and/or less than or equal to about 100 mol %, for example, less than or equal to about 90 mol %, less than or equal to about 89 mol %, less than or equal to about 88 mol %, less than or equal to about 87 mol %, less than or equal to about 86 mol %, or less than or equal to about 85 mol % based on the total content of the polymer.

For example, the polymer may further include additional structural units in addition to the aforementioned structural unit of Chemical Formula 1. In some example embodiments, the additional structural unit may be a structural unit represented by -D- included in the structural unit of Chemical Formula 1.

The terminal end of the polymer may be capped with an aryl group such as a phenyl group or a heterocyclic ring.

A weight average molecular weight of the polymer may be greater than or equal to about 5,000 Da, greater than or equal to about 10,000 Da, greater than or equal to about 15,000 Da, greater than or equal to about 20,000 Da, and greater than or equal to about 500,000 Da, and/or less than or equal to about 450,000 Da, less than or equal to about 400,000 Da, less than or equal to about 350,000 Da, less than or equal to about 300,000 Da, or less than or equal to about 200,000 Da.

The polymer has excellent electrical characteristics and flexibility and can therefore be used as an organic semiconductor material.

The organic semiconductor material may further include the polymer according to some example embodiments and any additional components required for preparing the organic semiconductor from the polymer. For example, such additional components may be a solvent, a binder, an elastomer, etc. for dissolving the polymer, and may further include additional components for imparting additional characteristics to the organic semiconductor produced.

The solvent may include, for example, a solvent selected to dissolve and/or disperse the polymer according to some example embodiments and to not react with the polymer. Such a solvent may be a solvent generally used for dissolving a polymer in the technical field to which the present invention pertains, and may be for example, an organic solvent having a relatively low boiling point which is heated and removed at a low temperature and leaves little solvent residue. Examples of such a solvent may be, for example, an aromatic or aliphatic hydrocarbon, a halogenated hydrocarbon, an ester, or an ether amide solvent, and specific examples thereof may include, for example, chloroform, tetrachloroethane, tetrahydrofuran, toluene, tetralin, decalin, anisole, xylene, ethyl acetate, methyl ethyl ketone, dimethyl formamide, chlorobenzene, dichlorobenzene, trichlorobenzene, propylene glycol monomethyl ether acetate (PGMEA), and a combination thereof, but is not limited thereto. In some example embodiments, the solvent may include xylene, toluene, tetralin, decalin, chloroform, chlorobenzene, ortho-dichlorobenzene, trichlorobenzene, or a combination thereof, but is not limited thereto.

The binder may improve dispersibility of the polymer according to some example embodiments, and may include, for example, polystyrene, etc., but is not limited thereto.

The elastomer maybe mixed with a polymer according to some example embodiments to improve stretchability of an organic semiconductor prepared therefrom, and may include, for example, an organic elastomer, an organic-inorganic elastomer, an inorganic elastomer-like material, or a combination thereof. Examples of the organic elastomer or organic/inorganic elastomer may include substituted or unsubstituted polyorganosiloxane such as polydimethylsiloxane; an elastomer including a substituted or unsubstituted butadiene moiety such as styrene-ethylene-butylene-styrene (SEBS), styrene-ethylene-propylene-styrene (SEPS), styrene-butadiene-styrene (SBS), styrene-isoprene-styrene (SIS), and styrene-isobutylene-styrene (SIBS); an elastomer containing a urethane moiety; an elastomer including an acrylic moiety; an elastomer including an olefin moiety, or a combination thereof, but are not limited thereto. The inorganic elastomer-like material may include, but is not limited to, ceramic, solid metal, liquid metal, or a combination thereof having elasticity and/or enhancing the elasticity of the organic semiconductor material.

The organic semiconductor material may be coated on a substrate or the like in a solution and/or suspension state to be manufactured into a thin film. The method of coating the solution and/or suspension on the substrate may include, for example spin-coating, dip-coating, screen printing, microcontact printing, doctor blading, and/or the like, but is not limited thereto.

As described above, the organic semiconductor material according to some example embodiments may be coated on a substrate and then dried and/or cured to form a thin film, and the thin film thus produced may be used as, for example, an organic semiconductor.

The aforementioned polymer or organic semiconductor material may be implemented as a thin film. The thin film may be a stretchable polymer thin film. The stretchable polymer thin film can flexibly respond to external forces or external movements such as twisting, pressing and pulling due to the stretching characteristics of the aforementioned polymer and can be easily restored to its original state.

An elastic modulus of the stretchable polymer thin film may be, for example, less than about 107 Pa, and within the above range, for example, may be about 10 Pa or more and less than about 107 Pa. For example, an elongation rate of the stretchable polymer thin film may be greater than or equal to about 10%, and within the above range, about 10% to about 1000%, about 10% to about 800%, about 10% to 500%, about 10% to about 300%, about 10% to about 200%, about 10% to about 100%, about 10% to about 90%, about 10% to about 80%, about 10% to about 70%, about 10% to about 60%, about 10% to about 50%, about 10% to about 40%, about 20% to about 70%, about 20% to about 60%, about 20% to about 50%, and/or about 20% to about 40%.

Herein, the elongation rate may be a percentage of a length change that is increased to a breaking point with respect to the initial length. For example, when the stretchable polymer thin film is stretched, a change in electrical properties of the stretchable polymer thin film may be relatively small. For example, when the stretchable polymer thin film is stretched by about 30%, the charge mobility change of the stretchable polymer thin film may be less than or equal to about 10%, less than or equal to about 8%, less than or equal to about 7%, less than or equal to about 5%, less than or equal to about 3%, less than or equal to about 2%, or less than or equal to about 1% and greater than or equal to about 0%, for example, greater than or equal to about 0.01%.

The stretchable polymer thin film may be a deposited thin film formed by vapor deposition or a coated thin film formed by a solution process. As described above, since the polymer and organic semiconductor material have good solubility in organic solvents, the stretchable polymer thin film may be a coated thin film formed by a solution process.

The stretchable polymer thin film may further include the aforementioned binder and/or the aforementioned elastomer in addition to the aforementioned polymer.

The binder may improve dispersibility of the aforementioned polymer, and may be, for example, polystyrene, but is not limited thereto.

The elastomer may be mixed with the aforementioned polymer to provide stretchability, and may include an organic elastomer, an inorganic elastomer, an inorganic elastomer-like material, or a combination thereof. Examples of the organic elastomer or organic/inorganic elastomer may include substituted or unsubstituted polyorganosiloxane such as polydimethylsiloxane; an elastomer including a substituted or unsubstituted butadiene moiety such as styrene-ethylene-butylene-styrene (SEBS), styrene-ethylene-propylene-styrene (SEPS), styrene-butadiene-styrene (SBS), styrene-isoprene-styrene (SIS), and styrene-isobutylene-styrene (SIBS); an elastomer containing a urethane moiety; an elastomer including an acrylic moiety; an elastomer including an olefin moiety, or a combination thereof, but are not limited thereto. The inorganic elastomer-like material may include, but is not limited to, ceramic, solid metal, liquid metal, or a combination thereof having elasticity and/or improving the elasticity of the stretchable polymer thin film.

As described above, the stretchable polymer thin film includes the first structural unit providing stretchability and the second structural unit providing good electrical characteristics, so that it can be stretched and have high charge mobility. Therefore, it can be applied to various electronic devices requiring stretchability and high charge mobility.

The electronic device may include, for example, an organic photoelectric device, an organic light emitting device, or an organic diode including an organic sensor; an organic thin film transistor; an attachable device such as a biometric sensor; or a device including them.

The electronic device may be applied to a bendable display panel, a foldable display panel, a rollable display panel, a wearable device, a skin-like display panel, a skin-like sensor, a large-area conformable display, smart clothing, and the like, but is not limited thereto.

Hereinafter, an example of a thin film transistor including the aforementioned polymer, organic semiconductor material, or stretchable polymer thin film will be described with reference to the drawings.

In the drawings, like reference numerals designate like elements throughout the specification. It will be understood that when an element such as a layer, film, region, or substrate is referred to as being “on” another element, it may be directly on the other element or intervening elements may also be present. In contrast, when an element is referred to as being “directly on” another element, there are no intervening elements present.

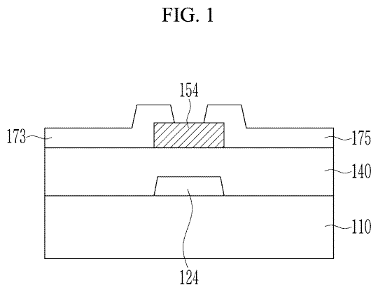

FIGS. 1 to 3 are cross-sectional views each showing a thin film transistor according to some example embodiments.

Referring to FIGS. 1 to 3, a thin film transistor according to some embodiments includes a gate electrode 124, an organic semiconductor 154 overlapped with the gate electrode 124, a gate insulating layer 140 between the gate electrode 124 and the organic semiconductor 154, and a source electrode 173 and a drain electrode 175 electrically connected to the organic semiconductor 154.

First, referring to FIG. 1, a thin film transistor according to some example embodiments may be a thin film transistor having a bottom gate and top contact structure. Specifically, a thin film transistor according to at least one embodiment includes a gate electrode 124 on the substrate 110; a gate insulating layer 140 on the gate electrode 124; an organic semiconductor 154 on the gate insulating layer 140; and a source electrode 173 and a drain electrode 175 electrically connected to the organic semiconductor 154.

The gate electrode 124 includes a conductive material disposed on a substrate 110 made of transparent glass, silicon, or polymer. The gate electrode 124 is connected to a gate line (not shown) that transmits the gate signal. The gate electrode 124 may be made of, for example, gold (Au), copper (Cu), nickel (Ni), aluminum (Al), molybdenum (Mo), chromium (Cr), tantalum (Ta), titanium (Ti), an alloy thereof, or a combination thereof, but is not limited thereto.

The gate insulating layer 140 is disposed on the gate electrode 124. The gate insulating layer 140 may be made of an organic material or an inorganic material. Examples of the organic material may include a soluble polymer compound such as a polyvinyl alcohol-based compound, a polyimide-based compound, a polyacryl-based compound, a polystyrene-based compound, benzocyclobutane (BCB), styrene-ethylene-butylene-styrene (SEBS), and the like, and examples of the inorganic material may include a silicon nitride (SiNx) and silicon oxide (SiO₂).

The organic semiconductor 154 is disposed on the gate insulating layer 140. The organic semiconductor 154 may include the aforementioned polymer or organic semiconductor material, and may be the aforementioned stretchable polymer thin film. The organic semiconductor 154 may be formed by a solution process such as spin coating, slit coating, or inkjet printing by preparing the aforementioned polymer or organic semiconductor material in a solution form. The organic semiconductor 154 may be formed by vacuum evaporation or thermal evaporation of the aforementioned polymer or organic semiconductor material.

The source electrode 173 and drain electrode 175 are disposed on the organic semiconductor 154. The source electrode 173 and the drain electrode 175 face the gate electrode 124 on the organic semiconductor 154 as the center. The source electrode 173 is connected to a data line (not shown) that transmits a data signal. The source electrode 173 and the drain electrode 175 may be made of gold (Au), copper (Cu), nickel (Ni), aluminum (Al), molybdenum (Mo), chromium (Cr), tantalum (Ta), titanium (Ti), an alloy thereof, or a combination thereof, but are not limited thereto.

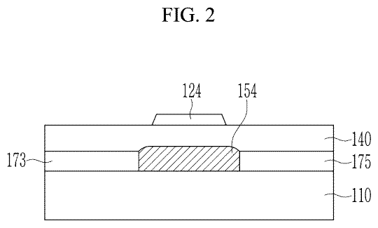

Referring to FIG. 2, the thin film transistor according to at least one embodiment may be a thin film transistor having a top gate and top contact structure, unlike the aforementioned embodiment. Specifically, a thin film transistor according to some example embodiments includes an organic semiconductor 154 on the substrate 110; a source electrode 173 and a drain electrode 175 electrically connected to the organic semiconductor 154; a gate insulating layer 140 on the organic semiconductor 154, the source electrode 173, and the drain electrode 175; and a gate electrode 124 on the gate insulating layer 140.

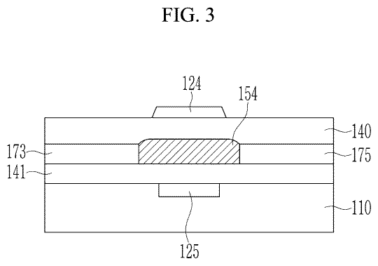

Referring to FIG. 3, the thin film transistor according to at least one embodiment may be a thin film transistor having a dual gate and bottom/top contact structure, unlike the aforementioned embodiment. Specifically, a thin film transistor according to at least one embodiment includes an organic semiconductor 154 on the substrate 110; a first gate electrode 125 positioned under the organic semiconductor 154; a second gate electrode 124 positioned on the organic semiconductor 154; a first gate insulating layer 141 between the organic semiconductor 154 and the first gate electrode 125; a second gate insulating layer 140 between the organic semiconductor 154 and the second gate electrode 124; and a source electrode 173 and a drain electrode 175 electrically connected to the organic semiconductor 154. The first gate electrode 125 may be buried in the substrate 110 or may be formed by impurity doping. The first gate electrode 125, the organic semiconductor 154, and the second gate electrode 124 may be overlapped with each other.

Although examples of thin film transistors have been described here, the present disclosure is not limited thereto and can be equally applied to thin film transistors of all structures.

The thin film transistor may be applied to various electronic devices as a switching device and/or a driving device. The electronic devices include, for example, a liquid crystal display device, an organic light emitting display device, a quantum dot display device, an electrophoretic display device, an organic photoelectric device, and an organic sensor, but is not limited thereto. The electronic device including the thin film transistor may be, for example, a flexible and stretchable electronic device, and may be a wearable device and/or a skin type device.

Hereinafter, the embodiments are illustrated in more detail with reference to examples. However, the following examples are for illustrative purposes only and do not limit the scope of rights.

### Synthesis of Polymers

### Synthesis Example 1: Synthesis of Polymer Having Structural Unit Represented by Chemical Formula A-1

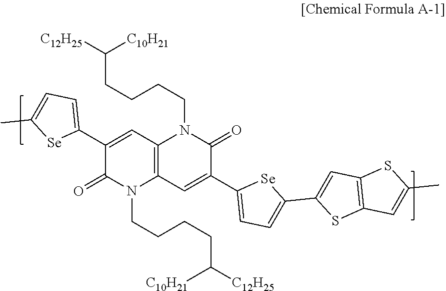

### Step 1: Synthesis of Monomer

The monomer is synthesized as follows.

### Step 1-1: Synthesis of 1-(5-decylheptadecyl)-6-methoxy-1,5-naphthyridin-2(1H)-one, Compound (2)

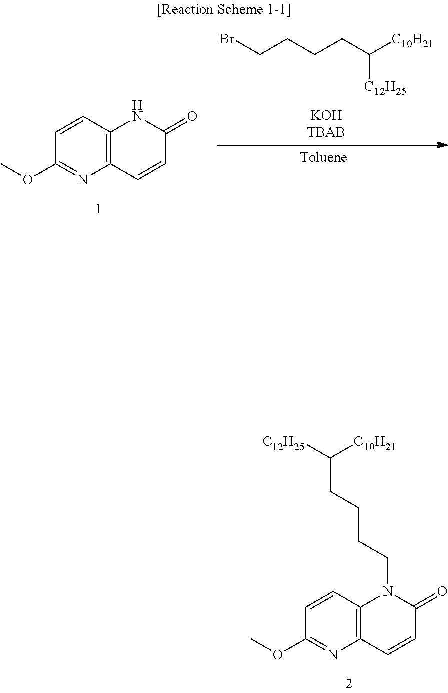

6-methoxy-1,5-naphthyridin-2(1H)-one (1 g, 5.68 mmol, Compound (1)), tetrabutylammonium bromide (TBAB) (0.366 g, 1.136 mmol), potassium hydroxide (KOH) (0.574 g, 10.224 mmol), and 11-(4-bromobutyl)tricosane) (7.38 g, 7.38 mmol) are dissolved in toluene (114 ml) and then, stirred at room temperature (24° C., hereinafter, applying the same) for 24 hours. After filtering, an organic layer is extracted therefrom by using ethyl acetate. Compound (2) in a yellow liquid state is obtained by using a silica gel column (1.53 g, Yield=48.54%).

¹H nuclear magnetic resonance (NMR) (300 MHz, chloroform d) δ (ppm): 7.75 (d, 2H), 7.61 (d, 2H), 6.96 (d, 2H), 6.87 (d, 2H), 4.22 (t, 2H), 3.99 (s, 3H), 1.66 (m, 2H), 1.26 (m, 48H), 0.88 (t, 3H).

### Step 1-2: Synthesis of 1-(5-decylheptadecyl)-1,5-naphthyridine-2,6(1H, 5H)-dione, Compound (3)

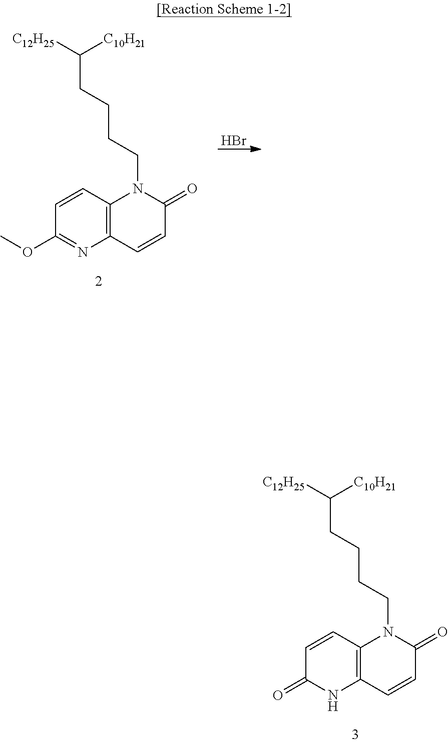

Compound (2) (1.53 g, 2.76 mmol) is dissolved in 48% HBr (6 ml) and then, stirred at 80° C. for 2 hours. After decreasing the temperature to room temperature, filtering is performed by using D.I. water. A yellow solid (1.42 g, Yield=95.29%) therefrom is used for the following reaction without additional purification.

¹H NMR (300 MHz, chloroform d) δ (ppm): 13.84 (s, 1H), 7.67 (d, 1H), 7.60 (d, 1H), 6.86 (dd, 2H), 6.87 (d, 2H), 4.20 (t, 2H), 1.68 (m, 2H), 1.25 (m, 48H), 0.87 (t, 3H).

### Step 1-3: Synthesis of 1,5-bis(5-decylheptadecyl)-1,5-dihydro-1,5-naphthyridine-2,6-dione, Compound (4)

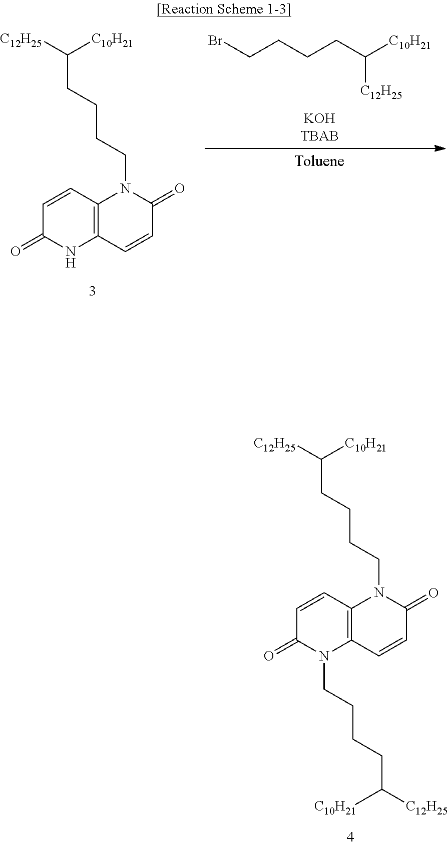

Compound (3) (0.8 g, 1.48 mmol), tetrabutylammonium bromide (0.097 g, 0.30 mmol), potassium hydroxide (0.15 g, 2.66 mmol), and 11-(4-bromobutyl)tricosane (0.88 g, 1.92 mmol) are dissolved in toluene (30 ml) and then, stirred at room temperature for 24 hours. After filtering, an organic layer is extracted therefrom by using ethyl acetate. Compound (4) in a yellow liquid state is obtained therefrom by using silica gel column. (0.35 g, Yield=25.72%)

¹H NMR (300 MHz, chloroform d) δ (ppm): 7.54 (d, 2H), 6.86 (d, 2H), 4.22 (t, 4H), 1.68 (m, 4H), 1.26 (m, 96H), 0.88 (t, 6H).

### Step 1-4: Synthesis of 3,7-dibromo-1,5-bis(5-decylheptadecyl)-1,5-dihydro-1,5-naphthyridine-2,6-dione, Compound (5)

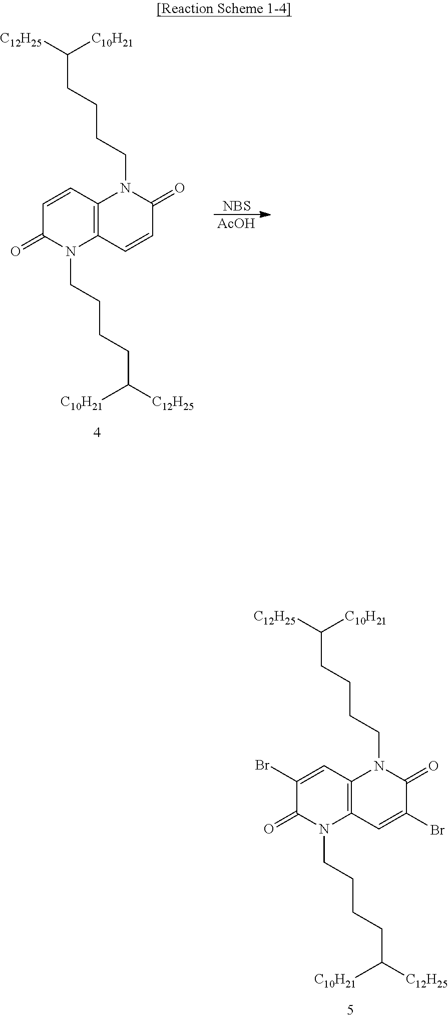

Compound (4) (0.382 g, 0.415 mmol) and NBS (0.207 g, 1.16 mmol) are dissolved in acetic acid (AcOH, 6.2 ml) and after blocking light, stirred at 90° C. for 24 hours. After removing AcOH, Compound (5) is obtained as a yellow solid through silica gel column. (0.247 g, Yield=55.24%)

¹H NMR (300 MHz, chloroform d) δ (ppm): 7.95 (s, 2H), 4.25 (t, 4H), 1.69 (m, 4H), 1.26 (m, 96H), 0.88 (t, 6H).

### Step 1-5: Synthesis of 1,5-bis(5-decylheptadecyl)-3,7-di(selenophen-2-yl)-1,5-dihydro-1,5-naphthyridine-2,6-dione, Compound (6)

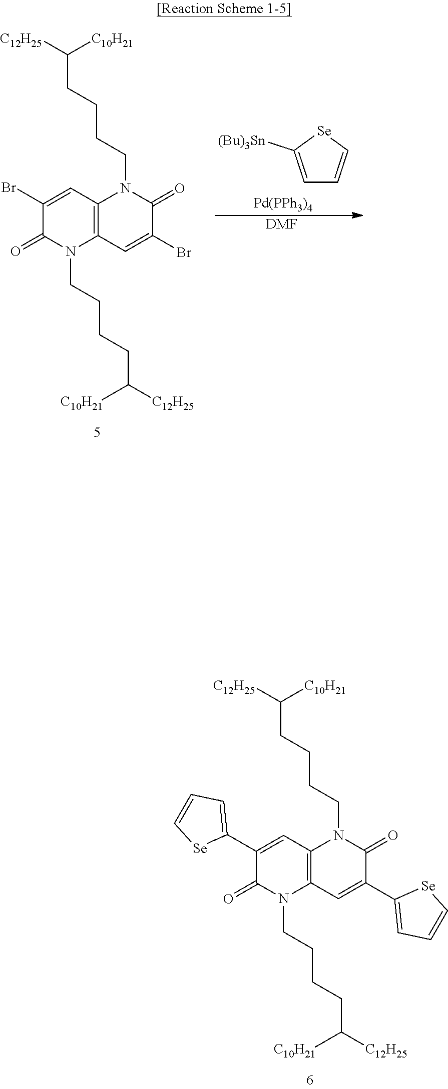

Compound (5) (0.44 g, 0.41 mmol), 2-(tributylstannyl)selenophene (0.52 g, 1.24 mmol), and Pd(PPh₃)₄ (tetrakis(triphenylphosphine)palladium (0) (0.022 g, 0.019 mmol) are dissolved in dimethyl formamide (DMF, 40 ml) and then, stirred at 130° C. for 12 hours. After removing the solvent, Compound (6) as an orange solid is obtained through silica gel column. (0.47 g, Yield=97.34%)

¹H NMR (300 MHz, chloroform d) δ (ppm): 8.31 (d, 2H), 8.06 (s, 2H), 7.90 (d, 4H), 7.46 (q, 2H), 4.17 (t, 4H), 1.84 (m, 4H), 1.26 (m, 96H), 0.88 (t, 6H).

### Step 1-6: Synthesis of 3,7-bis(5-bromoselenophen-2-yl)-1,5-bis(5-decylheptadecyl)-1,5-dihydro-1,5-naphthyridine-2,6-dione, Compound (7)

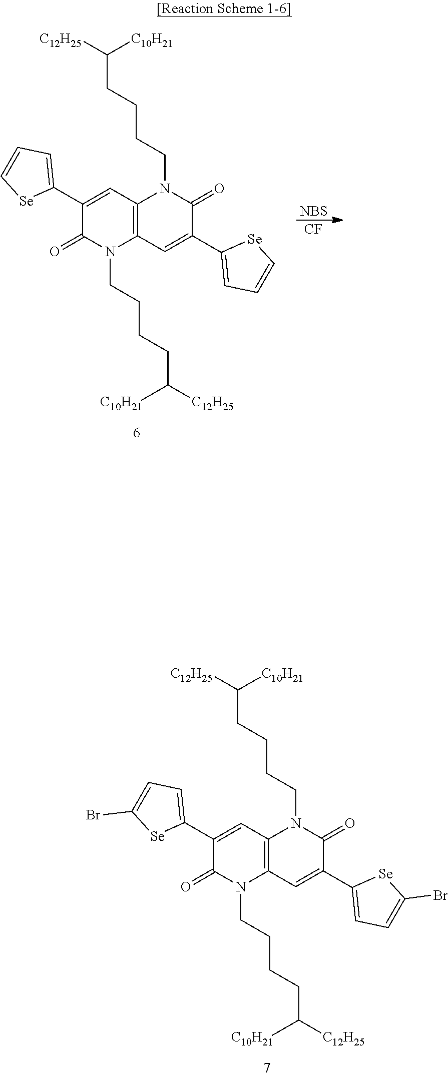

Compound (6) (0.467 g, 0.397 mmol) is dissolved in chloroform (CF, 30 ml) and then, blocked from light. N-Bromosucinimide (NBS) (0.155 g, 0.872 mmol) is added in portions thereto and then, stirred at room temperature for 12 hours. After adding D.I. water thereto, an organic layer is extracted therefrom through CF. After removing the solvent, Compound (7) as a red solid is obtained through silica gel column. (0.23 g, Yield=43.38%)

¹H NMR (300 MHz, chloroform d) δ (ppm): 7.98 (s, 2H), 7.58 (d, 2H), 7.39 (d, 2H), 4.44 (t, 4H), 1.80 (m, 2H), 1.26 (m, 96H), 0.88 (t, 6H).

### Step 2: Synthesis of Polymer A-1

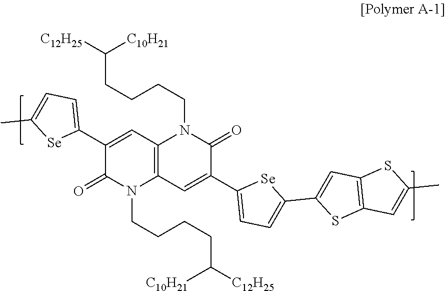

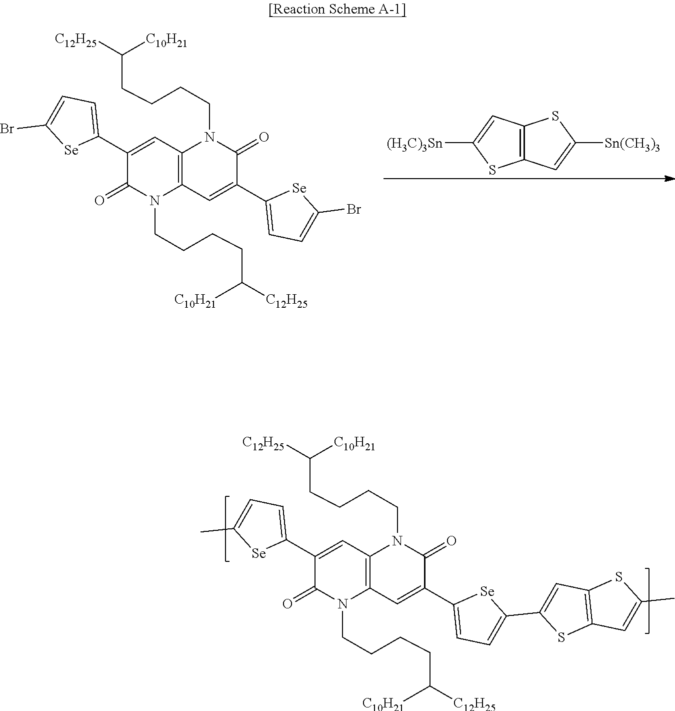

Compound (7) obtained in Step 1-6 of Synthesis Example 1 (3,7-bis(5-bromoselenophen-2-yl)-1,5-bis(5-decylheptadecyl)-1,5-dihydro-1,5-naphthyridine-2,6-dione, 133.5 mg, 0.1 mmol), 2,5-bis(trimethylstannyl)thieno[3,2-b]thiophene (46.6 mg, 0.1 mmol), Pd₂(dba)₃ (tris(dibenzylideneacetone)dipalladium (0), 1.83 mg, 0.002 mmol), and P(o-tol)₃ (tris(o-tolyl)phosphine, 2.43 mg, 0.008 mmol) are added to chlorobenzene (6 mL) and dissolved therein. The reactant is reacted under a nitrogen atmosphere at 115° C. for 48 hours. After the reaction, the reaction flask is cooled to room temperature, and the reactant is poured into an excessive amount of methanol for precipitation. A precipitate therefrom is filtered and Soxhlet-purified. The Soxhlet purification proceeds in an order of methanol-acetone-hexane-dichloromethane-chloroform, and a finally obtained solution in which the solid therefrom is dissolved in chloroform is concentrated and precipitated in methanol, obtaining Polymer A-1.

¹H NMR (500 MHz, CDCl₃) δ (ppm): 8.61-8.05 (broad, 2H), 7.48-6.95 (broad, 6H), 4.37-4.03 (broad, 4H), 2.24-1.24 (broad, 94H), 1.02-0.86 (broad, 12H).

### Synthesis Example 2: Synthesis of Polymer A-2

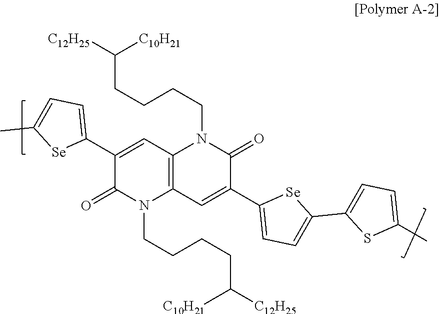

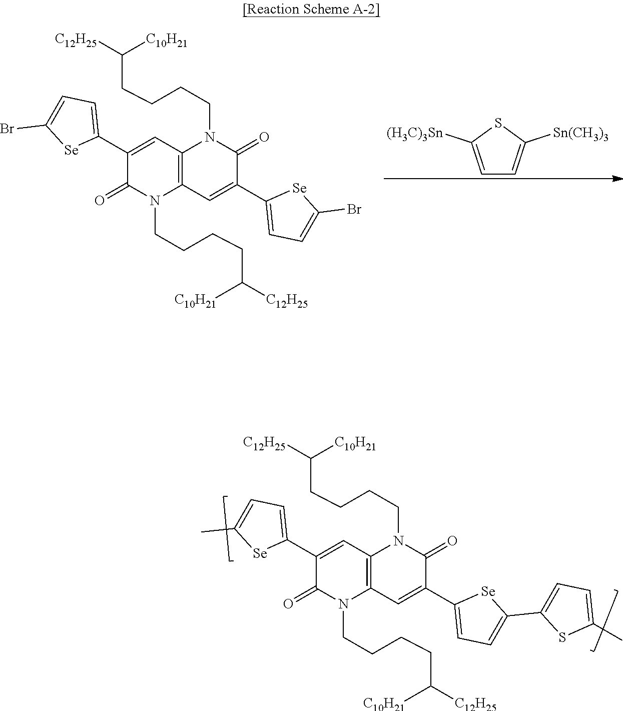

Compound (7) obtained in Step 1-6 of Synthesis Example 1 (3,7-bis(5-bromoselenophen-2-yl)-1,5-bis(5-decylheptadecyl)-1,5-dihydro-1,5-naphthyridine-2,6-dione, 133.5 mg, 0.1 mmol), 2,5-bis(trimethylstannyl)thiophene (40.9 mg, 0.1 mmol), Pd₂(dba)₃ (1.83 mg, 0.002 mmol), and P(o-tol)₃ (2.43 mg, 0.008 mmol) are added to 6 mL of chlorobenzene and dissolved therein. The reactant is reacted under a nitrogen atmosphere at 115° C. for 48 hours. After the reaction, the reaction flask is cooled to room temperature, and the reactant is poured into an excessive amount of methanol for precipitation. A precipitate therefrom is filtered and Soxhlet-purified. The Soxhlet purification proceeds in an order of methanol-acetone-hexane-dichloromethane-chloroform, and a finally obtained solution in which the solid therefrom is dissolved in chloroform is concentrated and precipitated in methanol, obtaining Polymer A-2.

¹H NMR (500 MHz, CDCl₃) δ (ppm): 8.82-8.43 (broad, 2H), 7.43-6.88 (broad, 6H), 4.35-4.08 (broad, 4H), 2.22-1.18 (broad, 94H), 1.02-0.80 (broad, 12H).

### Synthesis Example 3: Polymerization of Polymer A-3

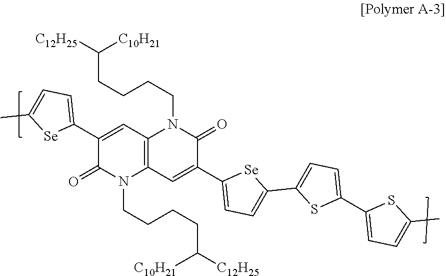

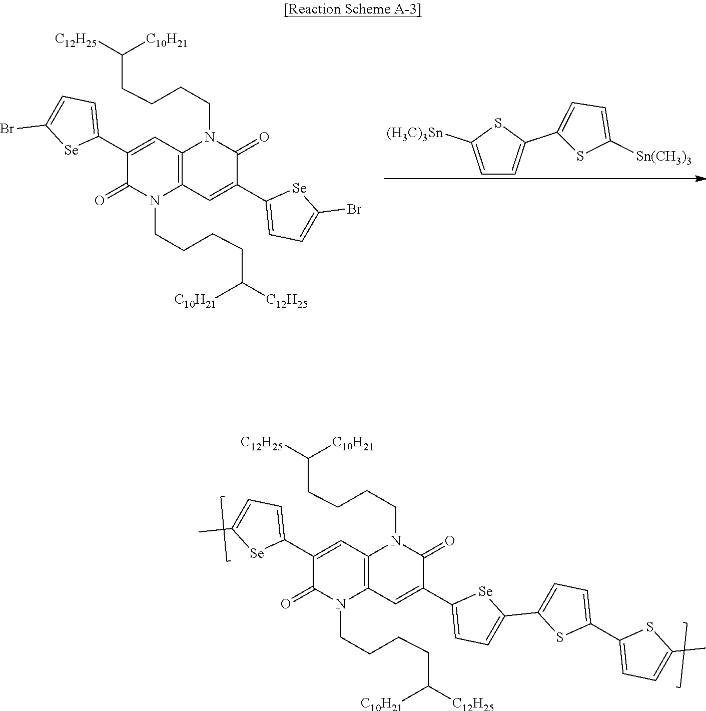

Compound (7) obtained in Step 1-6 of Synthesis Example 1 (3,7-bis(5-bromoselenophen-2-yl)-1,5-bis(5-decylheptadecyl)-1,5-dihydro-1,5-naphthyridine-2,6-dione, 133.5 mg, 0.1 mmol), 5,5′-bis(trimethylstannyl)-2,2′-bithiophene (49.2 mg, 0.1 mmol), Pd₂(dba)₃ (1.83 mg, 0.002 mmol), and P(o-tol)₃ (2.43 mg, 0.008 mmol) are added to 6 mL of chlorobenzene and dissolved therein. The reactant is reacted under a nitrogen atmosphere at 115° C. for 48 hours. After the reaction, the reaction flask is cooled to room temperature, and the reactant is poured into an excessive amount of methanol for precipitation. A precipitate therefrom is filtered and Soxhlet-purified. The Soxhlet purification proceeds in an order of methanol-acetone-hexane-dichloromethane-chloroform, and a finally obtained solution in which the solid therefrom is dissolved in chloroform is concentrated and precipitated in methanol, obtaining Polymer A-3.

¹H NMR (500 MHz, CDCl₃) δ (ppm): 8.71-8.23 (broad, 4H), 7.51-7.01 (broad, 6H), 4.33-4.08 (broad, 4H), 2.13-1.14 (broad, 94H), 1.05-0.83 (broad, 12H).

### Synthesis Example 4: Polymerization of Polymer A-4

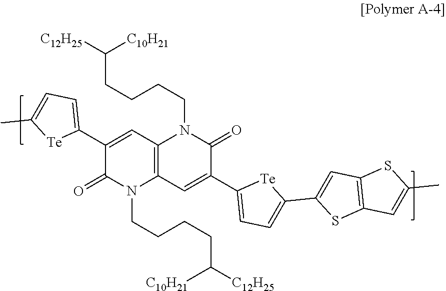

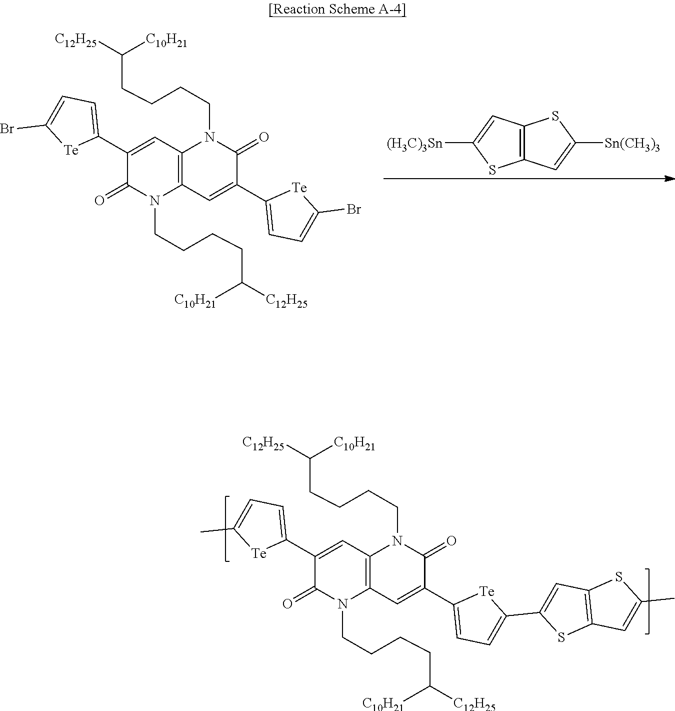

The compound obtained in the steps 1-5 and 1-6 of Synthesis Example 1 (3,7-bis(5-bromotellurophen-2-yl)-1,5-bis(5-decylheptadecyl)-1,5-dihydro-1,5-naphthyridine-2,6-dione, 143.3 mg, 0.1 mmol), 2,5-bis(trimethylstannyl)thieno[3,2-b]thiophene (46.6 mg, 0.1 mmol), Pd₂(dba)₃ (tris(dibenzylideneacetone)dipalladium (0), 1.83 mg, 0.002 mmol), and P(o-tol)₃ (tris(o-tolyl)phosphine, 2.43 mg, 0.008 mmol) are dissolved in 6 mL of chlorobenzene. The reactant is reacted under a nitrogen atmosphere at 115° C. for 48 hours. After the reaction, the reaction flask is cooled to room temperature, and the reactant is poured into an excessive amount of methanol for precipitation. A precipitate therefrom is filtered and Soxhlet-purified. The Soxhlet purification proceeds in an order of methanol-acetone-hexane-dichloromethane-chloroform, and a finally obtained solution in which the solid therefrom is dissolved in chloroform is concentrated and precipitated in methanol, obtaining Polymer A-4.

### Comparative Synthesis Example 1: Polymerization of Polymer B-1

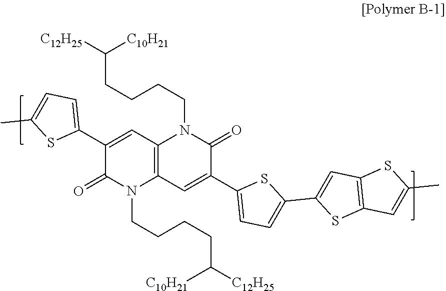

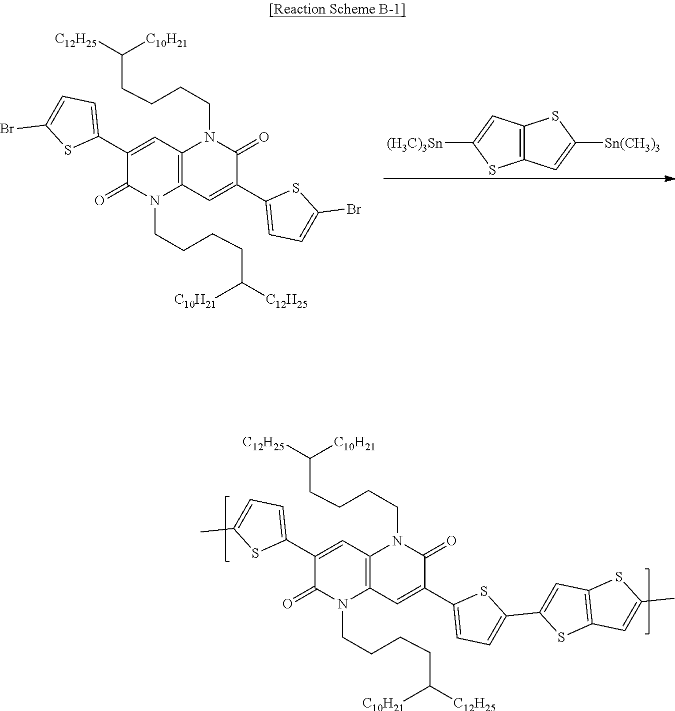

3,7-bis(5-bromothiophen-2-yl)-1,5-bis(5-decylheptadecyl)-1,5-dihydro-1,5-naphthyridine-2,6-dione (124.2 mg, 0.1 mmol), 2,5-bis(trimethylstannyl)thieno[3,2-b]thiophene (46.6 mg, 0.1 mmol), Pd₂(dba)₃ (1.83 mg, 0.002 mmol), and P(o-tol)₃ (2.43 mg, 0.008 mmol) are dissolved in 6 mL of chlorobenzene. The reactant is reacted under a nitrogen atmosphere at 115° C. for 48 hours. After the reaction, the reaction flask is cooled to room temperature, and the reactant is poured into an excessive amount of methanol for precipitation. A precipitate therefrom is filtered and Soxhlet-purified. The Soxhlet purification proceeds in an order of methanol-acetone-hexane-dichloromethane-chloroform, and a finally obtained solution in which the solid therefrom is dissolved in chloroform is concentrated and precipitated in methanol, obtaining Polymer B-1.

¹H NMR (500 MHz, CDCl₃) δ (ppm): 8.61-8.05 (broad, 2H), 7.49-6.90 (broad, 6H), 4.37-4.03 (broad, 4H), 2.24-1.24 (broad, 94H), 1.02-0.86 (broad, 12H).

### Manufacturing of Thin Film Transistor

### Example A-1

A substrate doped with Si at a relatively high concentration is used as a gate electrode, and a SiO₂ insulating layer (gate insulating layer, 300 nm) is formed on the gate electrode. After a surface treatment with octadecyltrimethoxysilane (OTS), Polymer A-1 of Synthesis Example 1 is added to chlorobenzene at a concentration of 0.5 wt % to obtain a polymer solution, and the polymer solution is spin-coated to be 300 Å thick at 1000 rpm and heat-treated at 130° C. for 1 hour under a nitrogen atmosphere to form an active layer (length: 50 μm, width: 1000 μm). On the active layer, Au is thermally deposited to form a source electrode and a drain electrode, manufacturing a thin film transistor.

### Example A-2

A thin film transistor is manufactured in the same manner as in Example A-1 except that the active layer is formed by using Polymer A-2 of Synthesis Example 2 instead of the polymer of Synthesis Example 1.

### Example A-3

A thin film transistor is manufactured in the same manner as in Example A-1 except that the active layer is formed by using Polymer A-3 of Synthesis Example 3 instead of the polymer of Synthesis Example 1.

### Example A-4

A thin film transistor is manufactured in the same manner as in Example A-1 except that the active layer is formed by using Polymer A-4 of Synthesis Example 4 instead of the polymer of Synthesis Example 1.

### Comparative Example B-1

A thin film transistor is manufactured in the same manner as in Example A-1 except that the active layer is formed by using Polymer B-1 of Comparative Synthesis Example 1 instead of the polymer of Synthesis Example 1.

### Evaluation I: Planarity of Compound

In the following polymers, a compound composed of a repeating unit of one of Chemical Formulas 1 to 3 is calculated with respect to a molecular skeleton of an energetically optimized structure through Gaussian09 SW (Method: DFT B3LYP Basis set: DGDZVP), and in the corresponding molecular skeleton, the shortest axis length (Z) is divided by the longest axis length (X) to obtain an aspect ratio (Z/X), and the results are shown in Table 1.

TABLE 1

| | Z length (Å) | X length (Å) | Aspect Ratio (Z/X) |
|---|---|---|---|
| Polymer B-1 | 9.61 | 23.47 | 0.409 |
| Polymer A-1 | 9.63 | 23.72 | 0.406 |
| Polymer A-4 | 9.73 | 24.01 | 0.405 |

Referring to Table 1, as a central element of a linker, which is a heterocyclic ring substituted on both sides of an NTD core, increases in a size, that is, as it is S, Se, and Te, the longest axis length X and the aspect ratio decrease. This means that molecular linearity of increases, wherein the increase in molecular linearity leads to improved molecular stacking ability and crystallinity and results in excellent charge mobility in the polymer thin films.

### Evaluation II: Charge Mobility Measurement

The charge mobility of the thin film transistors according to Examples A-1 to A-4 and Comparative Example B-1 is measured by using Semiconductor Characterization System (4200-SCS) made by Keithley. The results of Example A-1 and Comparative Example B-1 among them are provided in Table 2.

TABLE 2

| | Charge mobility | | | On-off |
|---|---|---|---|---|
| | (cm²/Vs) | V_{T} | | ratio |
| Example A-1 | 0.45 | −1.3 | V | 2.8 × 10⁷ |
| Comparative | 0.1 | 1.0 | V | 1.7 × 10⁵ |
| Example B-1 | | | | |

Referring to Table 2, the thin film transistor of Example A-1 exhibits excellent charge mobility, compared with the thin film transistor of Comparative Example B-1. While this disclosure has been described in connection with what is presently considered to be practical example embodiments, it is to be understood that the invention is not limited to the disclosed embodiments. On the contrary, it is intended to cover various modifications and equivalent arrangements included within the spirit and scope of the appended claims.

What is claimed is:

1. A polymer comprising:
a structural unit represented by Chemical Formula 1 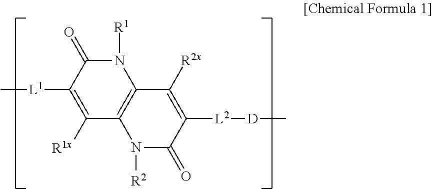
wherein, in Chemical Formula 1,
R¹ and R² are each independently a substituted or unsubstituted C1 to C50 alkyl group, a substituted or unsubstituted C1 to C50 alkoxy group, a substituted or unsubstituted C6 to C50 aryl group, a substituted or unsubstituted C2 to C50 heteroaryl group, a substituted or unsubstituted C3 to C50 cycloalkyl group, a substituted or unsubstituted C3 to C50 heterocycloalkyl group, a substituted or unsubstituted C7 to C50 aralkyl group, —COR^{a}, —OC(═O)R^{b}, —C(═O)OR^{c}, —OC(═O)OR^{d}, —NR^{e}R^{f}, a cyano group, or a combination thereof,
R^{a} to R^{f} are each independently hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a cyano group, or a combination thereof,
R^{1x} and R^{2x} are each independently hydrogen, deuterium, or a C1 to C10 alkyl group,
L¹ and L² are each independently a substituted or unsubstituted Se-containing aromatic ring, a substituted or unsubstituted Te-containing aromatic ring, or a fused ring thereof, and
D is a substituted or unsubstituted C6 to C30 arylene group; a substituted or unsubstituted divalent C3 to C30 heterocyclic group including at least one selected from N, O, S, Se, Te, and Si; a fused ring thereof; or a combination thereof.

2. The polymer of claim 1, wherein D is at least one substituted or unsubstituted phenylene group; at least one substituted or unsubstituted naphthylene group; at least one substituted or unsubstituted anthracenylene group; at least one substituted or unsubstituted phenanthrenylene group; at least one substituted or unsubstituted pentagonal rings including at least one of N, O, S, Se, Te, or Si; a fused ring of two or more of the substituted or unsubstituted pentagonal rings; a fused ring of at least one of the substituted or unsubstituted pentagonal rings and at least one substituted or unsubstituted phenylene group; a fused ring of at least one substituted or unsubstituted pentagonal ring and at least one substituted or unsubstituted naphthylene group; a fused ring of at least one substituted or unsubstituted pentagonal ring and at least one substituted or unsubstituted anthracenylene group a fused ring of at least one substituted or unsubstituted pentagonal ring and at least one substituted or unsubstituted phenanthrenylene group; or a combination thereof.

3. The polymer of claim 1, wherein D is one of an electron donating moiety listed in Group 1: 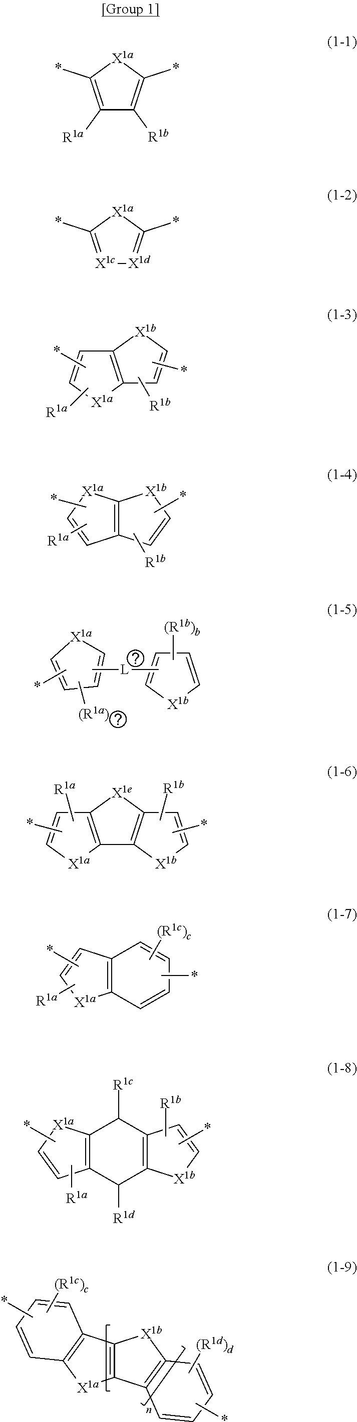 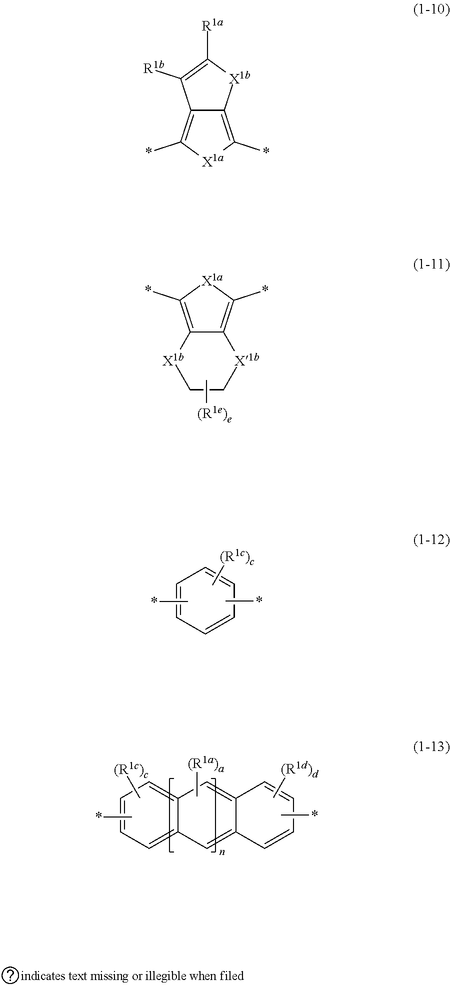
wherein, in Group 1,
X^{1a}, X^{1b}, and X′^{1b} are each independently O, S, Se, or Te,
X^{1c} and X^{1d} are N, CR^{x}, or SiR^{y},
X^{1e} is O, S, Se, Te, NR^{v}, CR^{w}R^{x}, or SiR^{y}R^{z},
R^{1a}, R^{1b}, R^{1c}, R^{1d}, R^{1e}, R^{x}, R^{y}, R^{v}, R^{w}, and R^{z} are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C3 to C30 heteroaryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group, or a combination thereof,
L^{a} is a single bond or a substituted or unsubstituted C2 to C10 alkenylene group
a and b each independently represent an integer from 1 to 2,
c and d each independently represent an integer from 1 to 3,
e represents an integer from 1 to 4
n represents 0, 1, or 2, and
* represents a linking point with a main chain of Chemical Formula 1.

4. The polymer of claim 1, wherein L¹ and L² are each independently selected from Group 2: 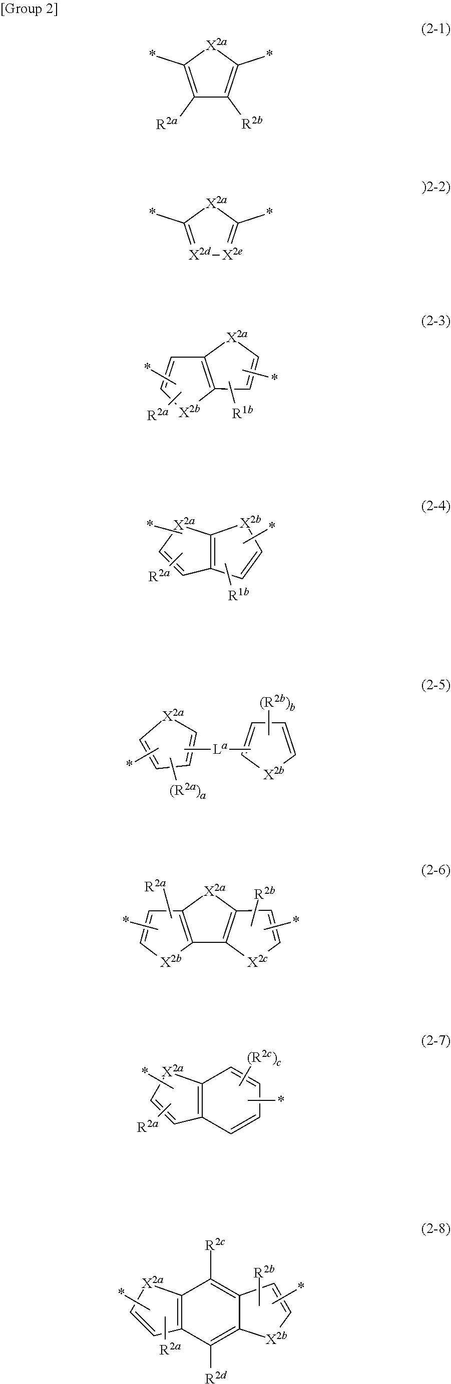
wherein, in Group 2,
X^{2a} is Se or Te,
X^{2b} and X^{2c} are each independently O, S, Se, or Te,
X^{2d} and X^{2e} are each independently N, CR^{x}, or SiR^{y},
R^{2a}, R^{2b}, R^{2c}, R^{2d}, R^{x}, and R^{y} are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C3 to C30 heteroaryl group, a substituted or unsubstituted C7 to C30 alkylaryl group, a substituted or unsubstituted C6 to C30 aryloxy group, a substituted or unsubstituted C3 to C30 heterocyclic group, a cyano group, or a combination thereof,
L^{a} is a single bond or a substituted or unsubstituted C2 to C10 alkenylene group,
a and b each independently represent an integer from 1 to 2,
c represents an integer from 1 to 3, and
* represents a linking point with a main chain of Chemical Formula 1.

5. The polymer of claim 1, wherein L¹ and L² are each independently a substituted or unsubstituted selenophene, a substituted or unsubstituted tellurophene, or a fused ring thereof.

6. The polymer of claim 1, wherein R¹ and R² are each independently a substituted or unsubstituted C6 to C30 linear alkyl group, a substituted or unsubstituted C6 to C30 branched alkyl group, or a combination.

7. The polymer of claim 1, wherein the polymer including the structural unit represented by Chemical Formula 1 is a polymer including a structural unit represented by at least one of Group 3-1:

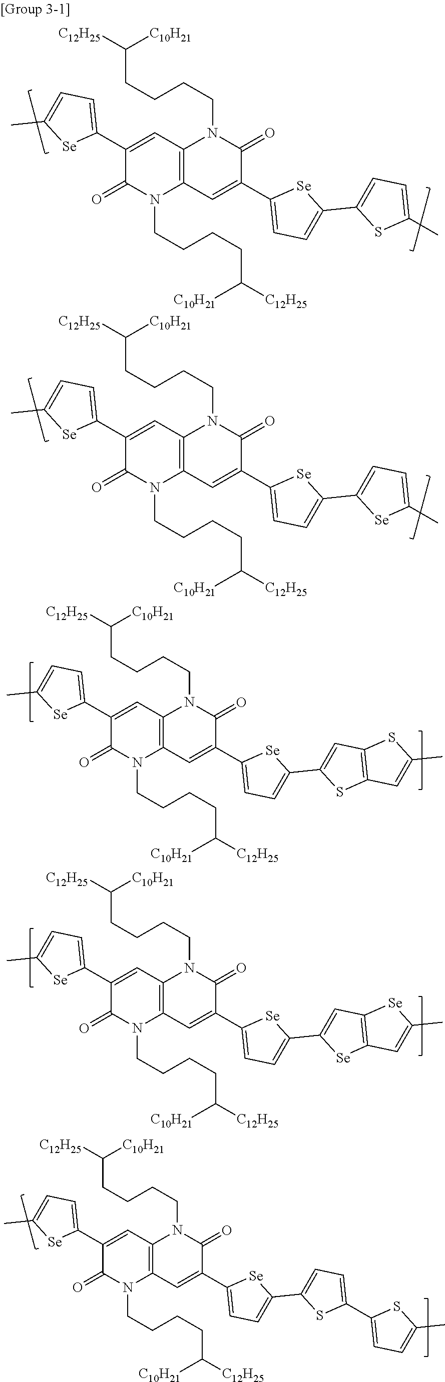

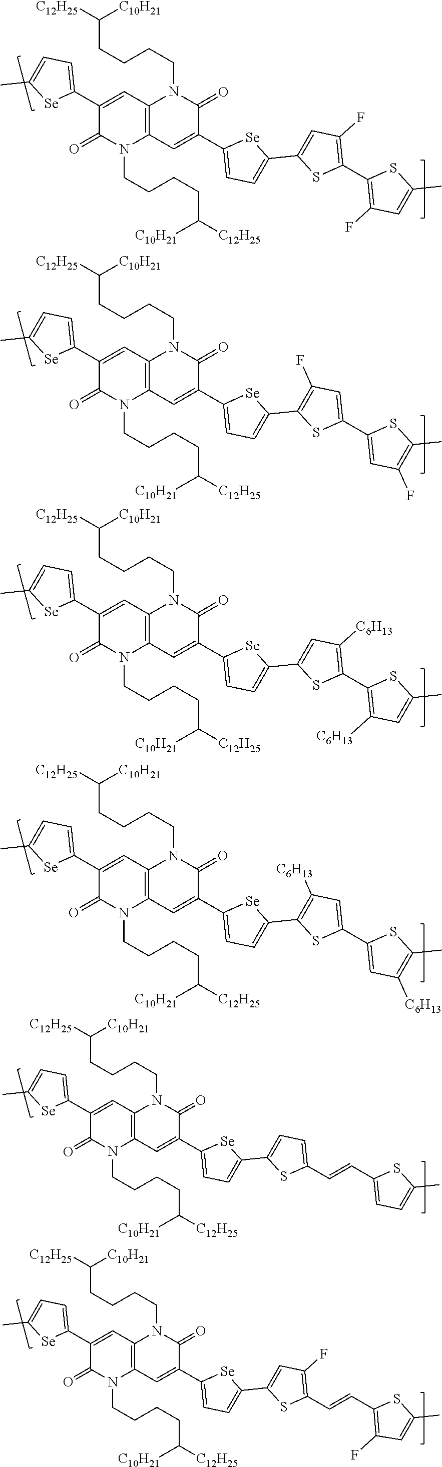

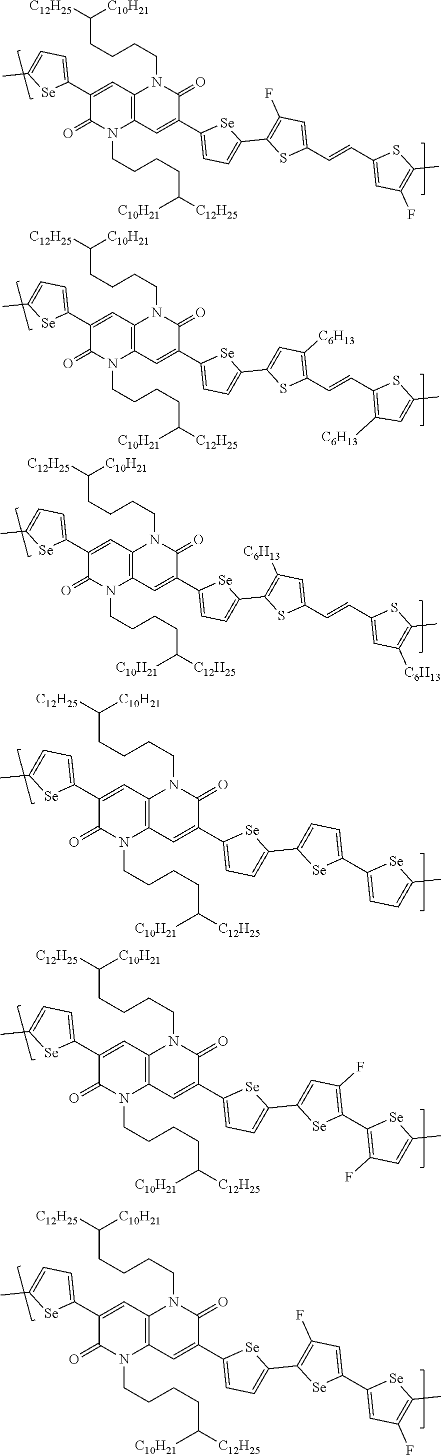

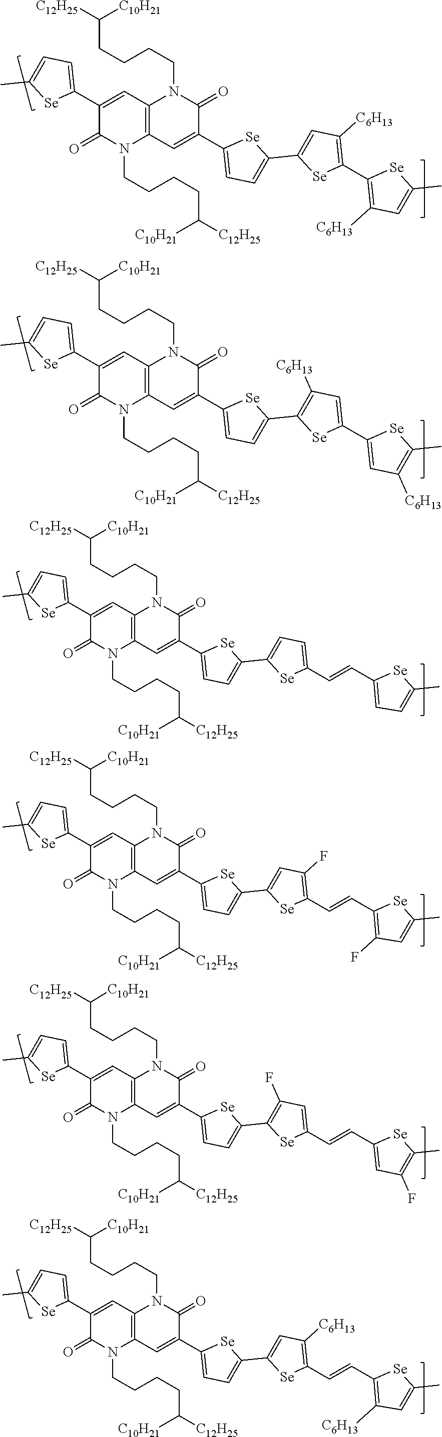

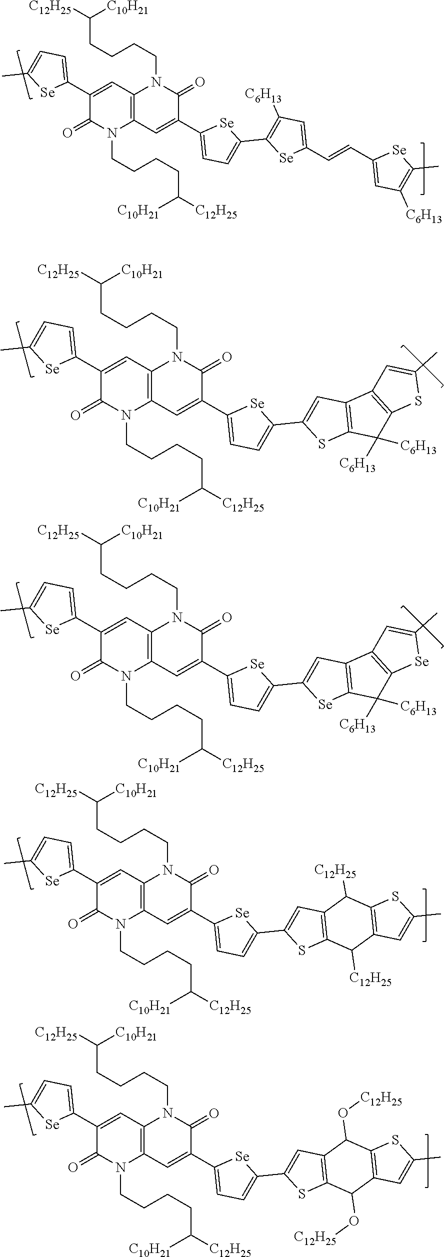

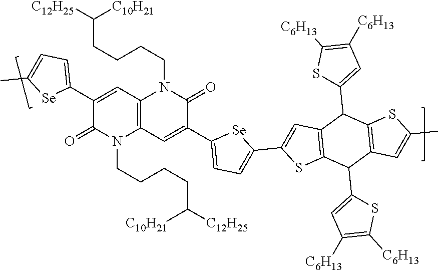

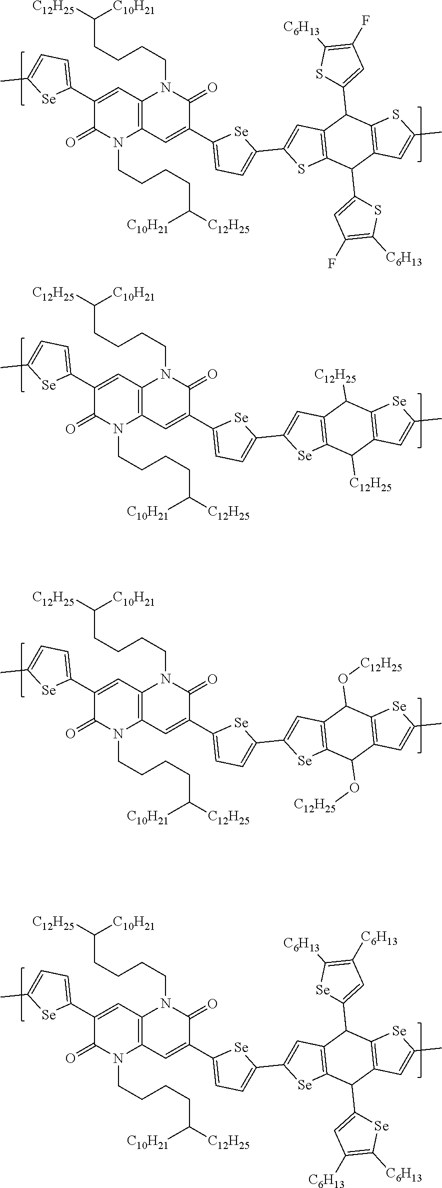

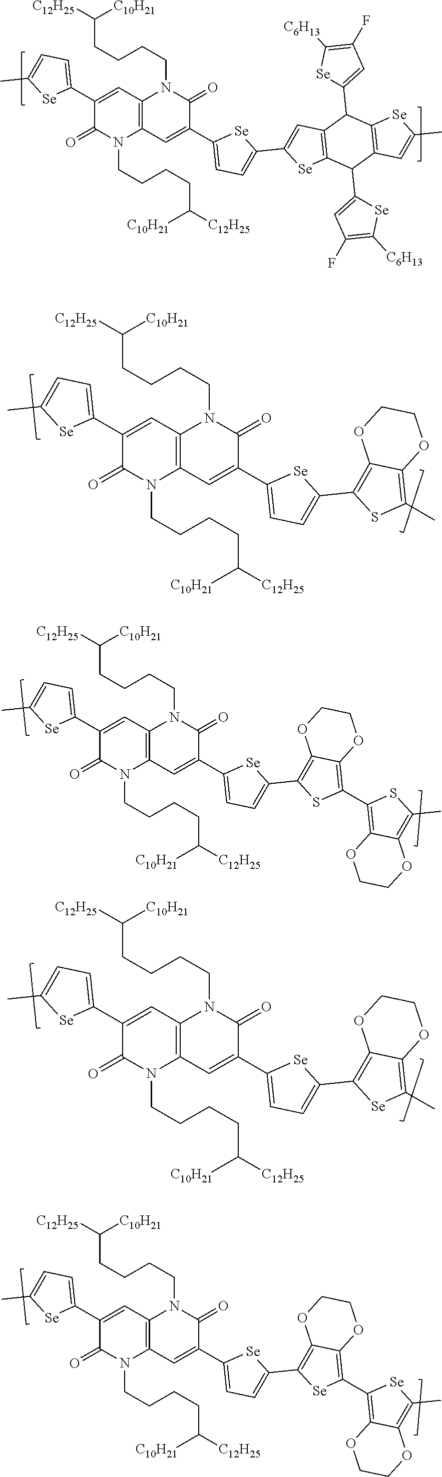

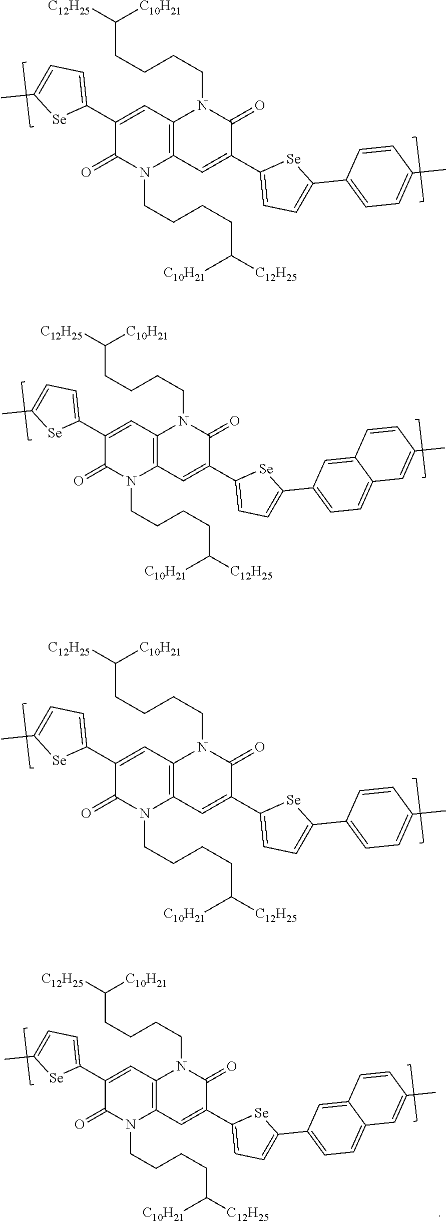

8. The polymer of claim 1, wherein
the polymer including the structural unit represented by Chemical Formula 1 is a polymer including a structural unit represented by at least one of Group 3-2:

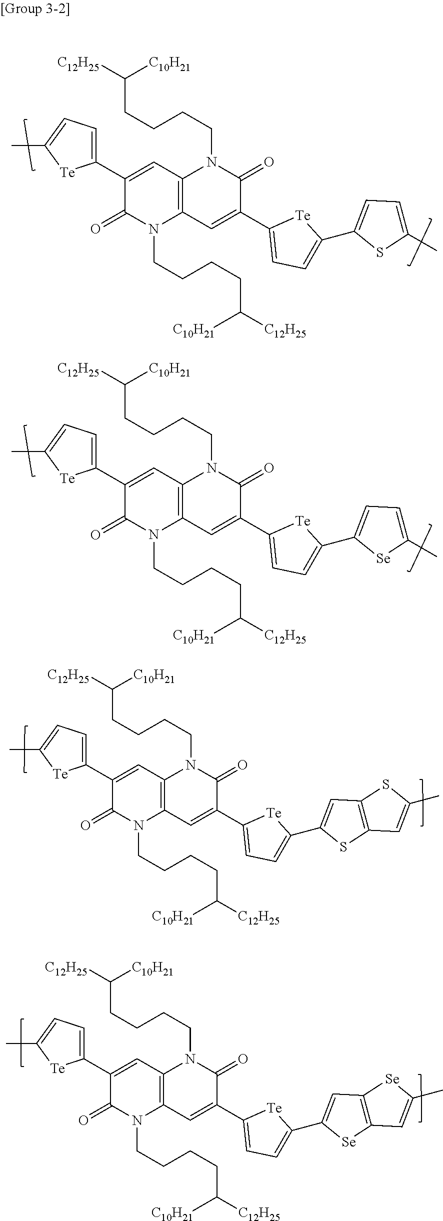

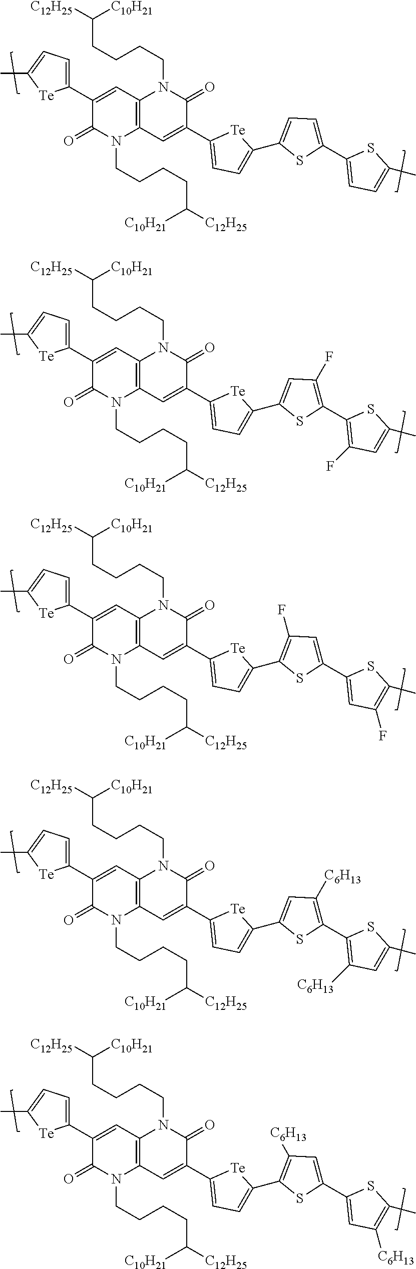

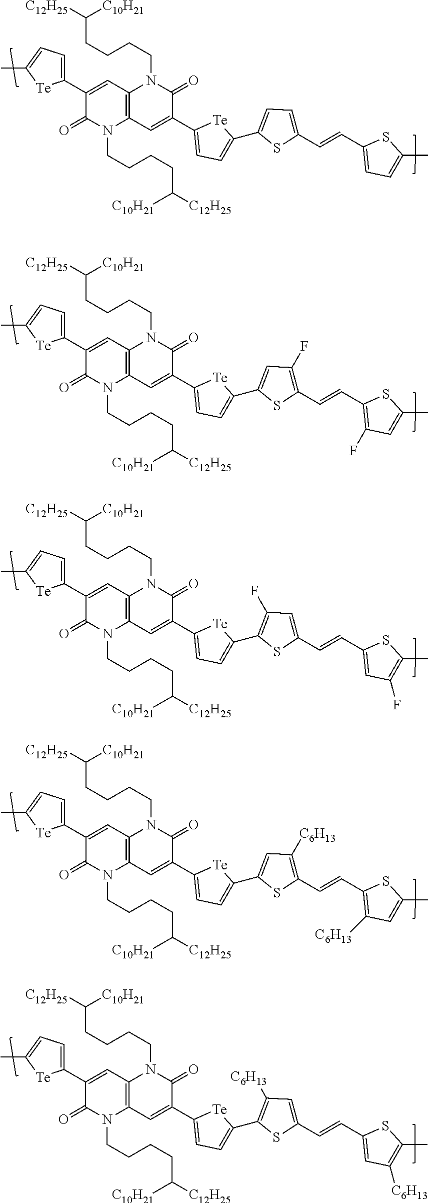

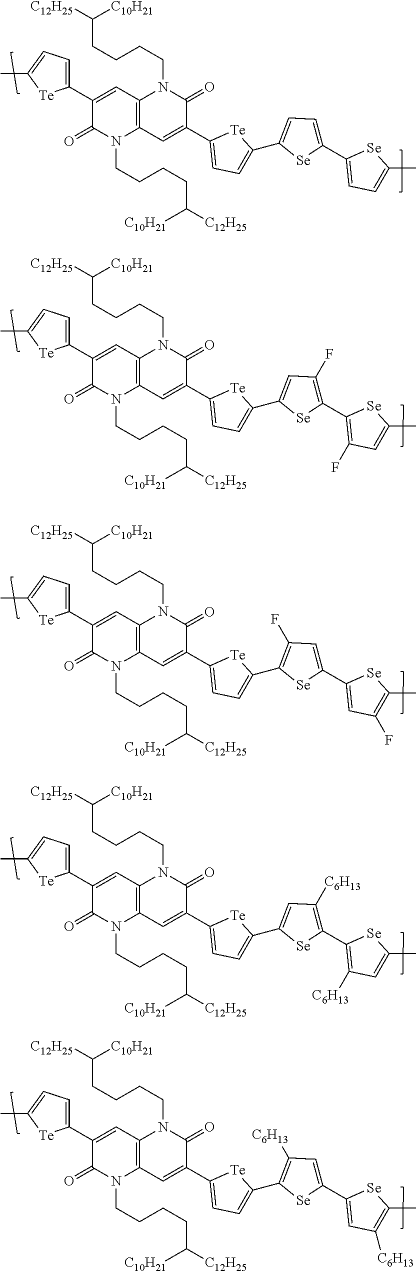

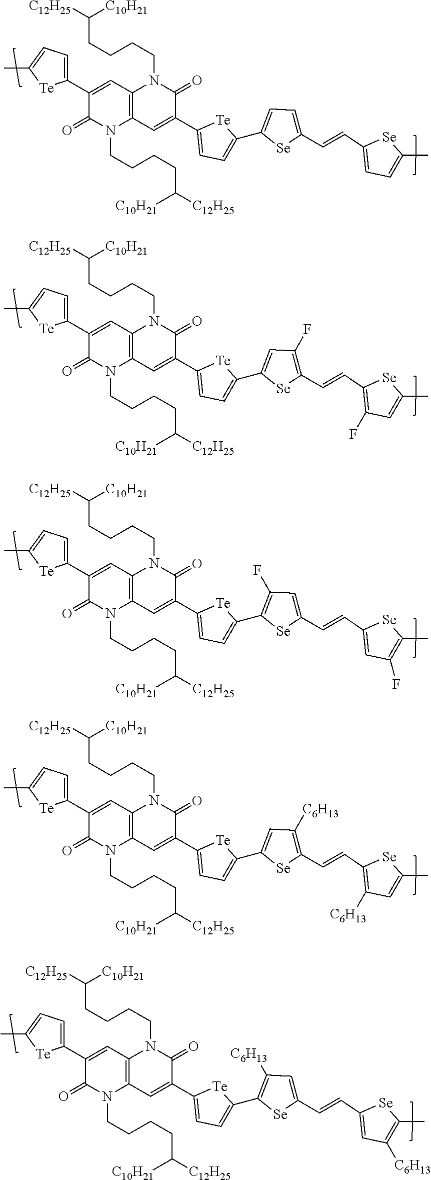

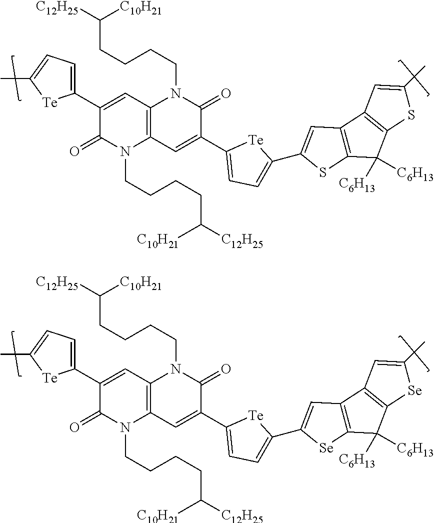

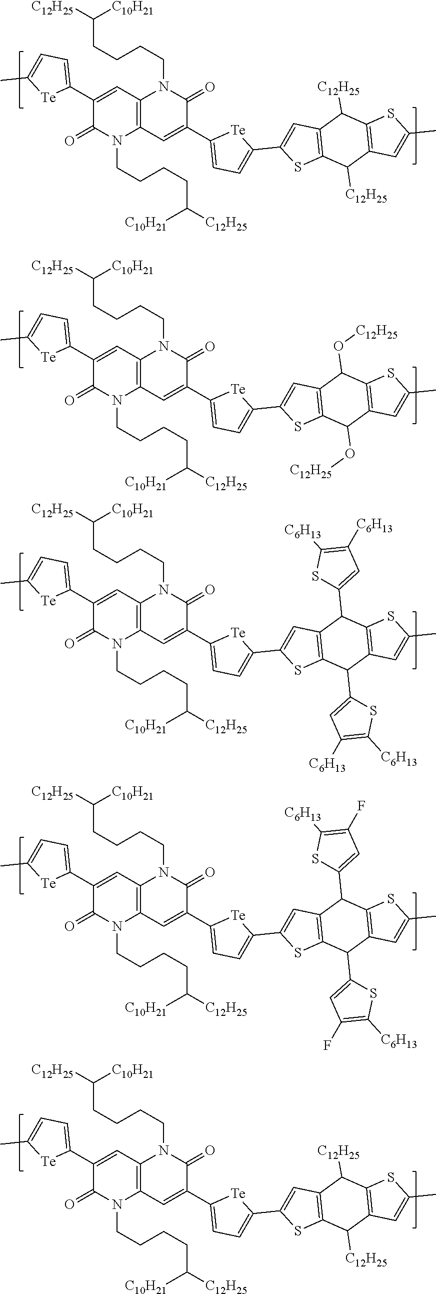

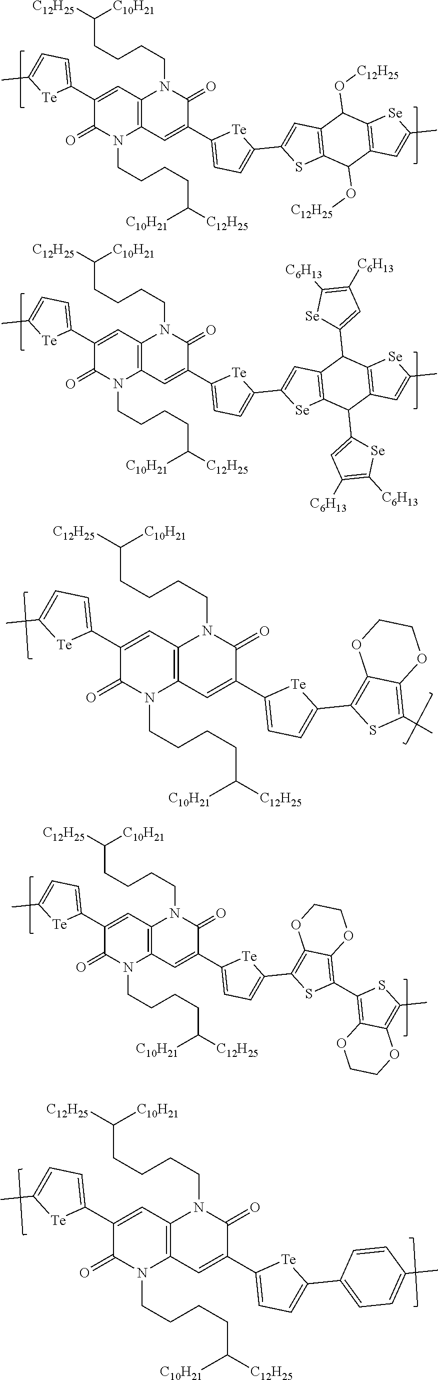

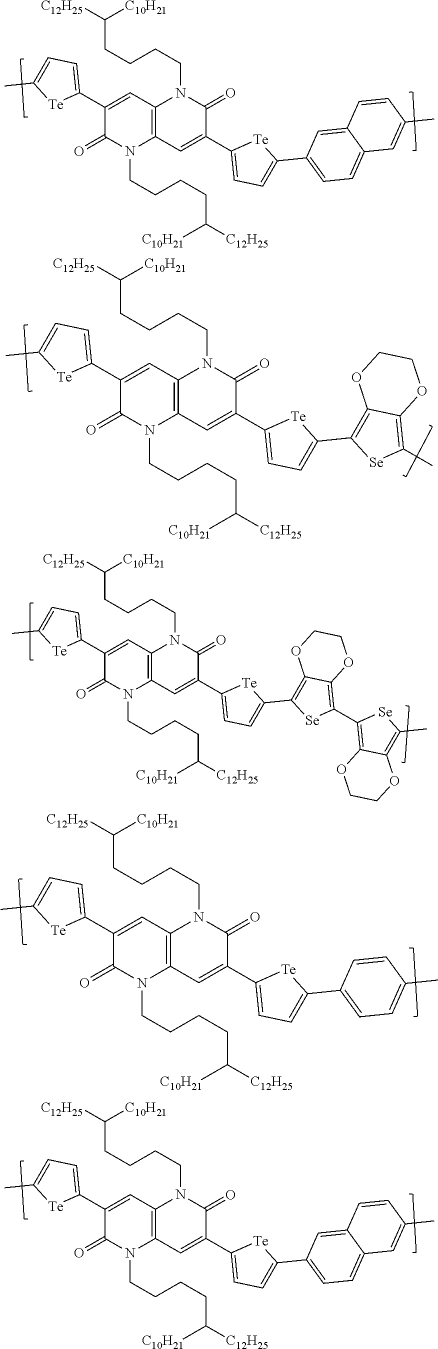

9. The polymer of claim 1, wherein,
in the polymer, an aspect ratio (Z/X) obtained by dividing a length of the shortest axis (Z) of the structural unit of Chemical Formula 1 by a length of the longest axis (X) is less than or equal to about 0.407.

10. An organic semiconductor material comprising the polymer of claim 1.

11. A stretchable polymer thin film comprising the polymer of claim 1.

12. The stretchable polymer thin film of claim 11 further comprising:
an elastomer.

13. The stretchable polymer thin film of claim 12, wherein a change in charge mobility is less than or equal to about 10% when the stretchable polymer thin film is stretched by about 30%.

14. A thin film transistor, comprising
a gate electrode,
an organic semiconductor overlapped with the gate electrode, and
a source electrode and a drain electrode electrically connected to the semiconductor,
wherein the organic semiconductor comprises a polymer comprising
a structural unit represented by Chemical Formula 1: 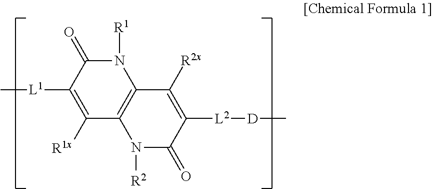
wherein, in Chemical Formula 1,
R¹ and R² are each independently a substituted or unsubstituted C1 to C50 alkyl group, a substituted or unsubstituted C1 to C50 alkoxy group, a substituted or unsubstituted C6 to C50 aryl group, a substituted or unsubstituted C2 to C50 heteroaryl group, a substituted or unsubstituted C3 to C50 cycloalkyl group, a substituted or unsubstituted C3 to C50 heterocycloalkyl group, a substituted or unsubstituted C7 to C50 aralkyl group, —COR^{a}, —OC(═O)R^{b}, —C(═O)OR^{c}, —OC(═O)OR^{d}, —NR^{e}R^{f}, a cyano group, or a combination thereof,
R^{a} to R^{f} are each independently hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a cyano group, or a combination thereof,
R^{1x} and R^{2x} are each independently hydrogen, deuterium, or a C1 to C10 alkyl group,
L¹ and L² are each independently a substituted or unsubstituted Se-containing aromatic ring, a substituted or unsubstituted Te-containing aromatic ring, or a fused ring thereof, and
D is a substituted or unsubstituted C6 to C30 arylene group; a substituted or unsubstituted divalent C3 to C30 heterocyclic group including at least one selected from N, O, S, Se, Te, and Si; a fused ring thereof; or a combination thereof.

15. The thin film transistor of claim 14, wherein D is at least one substituted or unsubstituted phenylene group; at least one substituted or unsubstituted naphthylene group; at least one substituted or unsubstituted anthracenylene group; at least one substituted or unsubstituted phenanthrenylene group; at least one substituted or unsubstituted pentagonal rings including at least one of N, O, S, Se, Te, or Si; a fused ring of two or more of the substituted or unsubstituted pentagonal rings; a fused ring of at least one of the substituted or unsubstituted pentagonal rings and at least one substituted or unsubstituted phenylene group; a fused ring of at least one substituted or unsubstituted pentagonal ring and at least one substituted or unsubstituted naphthylene group; a fused ring of at least one substituted or unsubstituted pentagonal ring and at least one substituted or unsubstituted anthracenylene group a fused ring of at least one substituted or unsubstituted pentagonal ring and at least one substituted or unsubstituted phenanthrenylene group; or a combination thereof.

16. The thin film transistor of claim 14, wherein D is one of an electron donating moiety listed in Group 1: 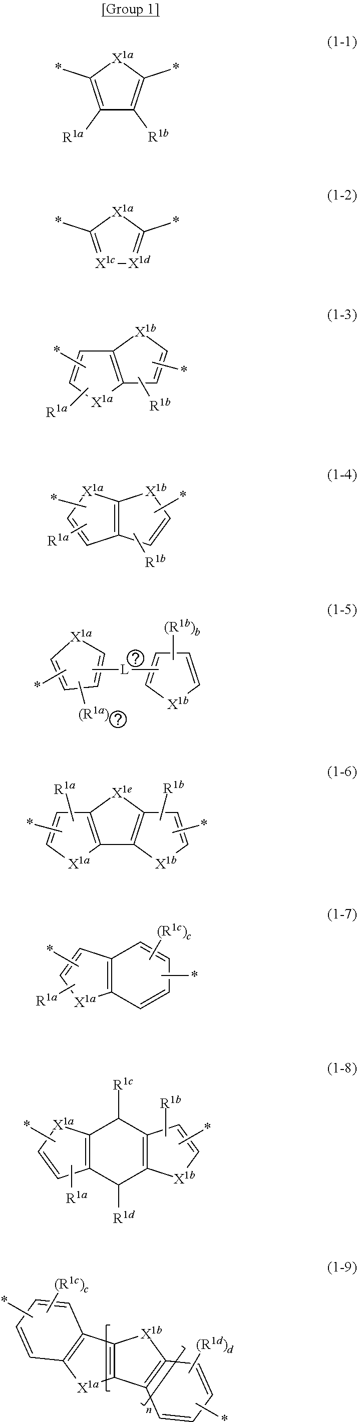 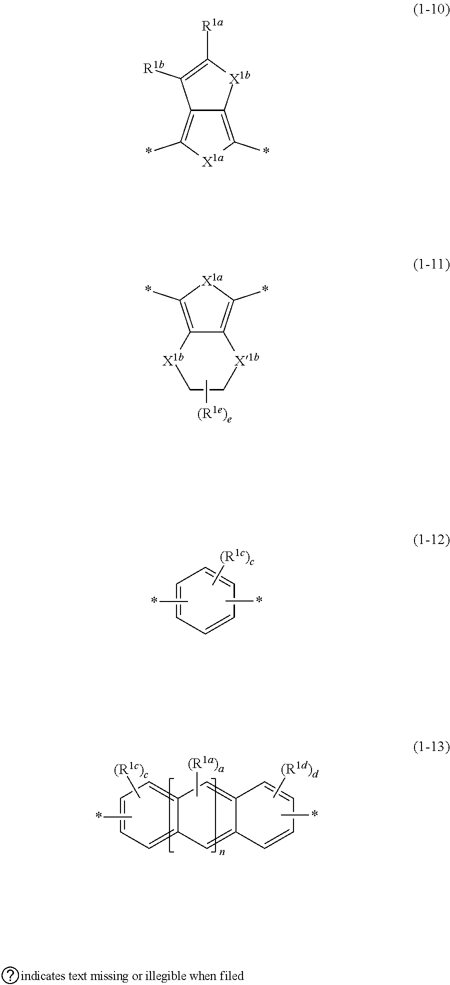
wherein, in Group 1,
X^{1a}, X^{1b}, and X′^{1b} are each independently O, S, Se, or Te,
X^{1c} and X^{1d} are N, CR^{x}, or SiR^{y},
X^{1e} is O, S, Se, Te, NR^{v}, CR^{w}R^{x}, or SiR^{y}R^{z}, R^{1a}, R^{1b}, R^{1c}, R^{1d}, R^{1e}, R^{x}, R^{y}, R^{v}, R^{w}, and R^{z} are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C3 to C30 cycloalkyl group, a substituted or unsubstituted C3 to C30 cycloalkenyl group, a substituted or unsubstituted C1 to C30 alkoxy group, a substituted or unsubstituted C3 to C30 cycloheteroalkyl group, a substituted or unsubstituted C3 to C30 cycloheteroalkenyl group, a substituted or unsubstituted C6 to C30 aryl group, a substituted or unsubstituted C3 to C30 heteroaryl group, a substituted or unsubstituted C7 to C30 alkylaryl group, a substituted or unsubstituted C6 to C30 aryloxy group, a substituted or unsubstituted C3 to C30 heterocyclic group, a halogen, or a cyano group, or a combination thereof,
L^{a} is a single bond or a substituted or unsubstituted C2 to C10 alkenylene group
a and b each independently represent an integer from 1 to 2,
c and d each independently represent an integer from 1 to 3,
e represents an integer from 1 to 4
n represents 0, 1, or 2, and
* represents a linking point with a main chain of Chemical Formula 1.

17. An electronic device comprising
a thin film transistor, comprising
a gate electrode,
an organic semiconductor overlapped with the gate electrode, and
a source electrode and a drain electrode electrically connected to the semiconductor,
wherein the organic semiconductor comprises a polymer comprising
a structural unit represented by Chemical Formula 1: 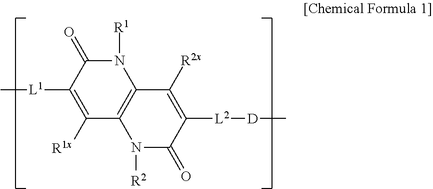
wherein, in Chemical Formula 1,
R¹ and R² are each independently a substituted or unsubstituted C1 to C50 alkyl group, a substituted or unsubstituted C1 to C50 alkoxy group, a substituted or unsubstituted C6 to C50 aryl group, a substituted or unsubstituted C2 to C50 heteroaryl group, a substituted or unsubstituted C3 to C50 cycloalkyl group, a substituted or unsubstituted C3 to C50 heterocycloalkyl group, a substituted or unsubstituted C7 to C50 aralkyl group, —COR^{a}, —OC(═O)R^{b}, —C(═O)OR^{c}, —OC(═O)OR^{d}, —NR^{e}R^{f}, a cyano group, or a combination thereof,
R^{a} to R^{f} are each independently hydrogen, a substituted or unsubstituted C1 to C30 alkyl group, a substituted or unsubstituted C1 to C30 alkoxy group, a substituted or unsubstituted C6 to C30 aryl group, a substituted or unsubstituted C2 to C30 heteroaryl group, a cyano group, or a combination thereof,
R^{1x} and R^{2x} are each independently hydrogen, deuterium, or a C1 to C10 alkyl group,
L¹ and L² are each independently a substituted or unsubstituted Se-containing aromatic ring, a substituted or unsubstituted Te-containing aromatic ring, or a fused ring thereof, and
D is a substituted or unsubstituted C6 to C30 arylene group; a substituted or unsubstituted divalent C3 to C30 heterocyclic group including at least one selected from N, O, S, Se, Te, and Si; a fused ring thereof; or a combination thereof.

18. The electronic device of claim 17, wherein D is at least one substituted or unsubstituted phenylene group; at least one substituted or unsubstituted naphthylene group; at least one substituted or unsubstituted anthracenylene group; at least one substituted or unsubstituted phenanthrenylene group; at least one substituted or unsubstituted pentagonal rings including at least one of N, O, S, Se, Te, or Si; a fused ring of two or more of the substituted or unsubstituted pentagonal rings; a fused ring of at least one of the substituted or unsubstituted pentagonal rings and at least one substituted or unsubstituted phenylene group; a fused ring of at least one substituted or unsubstituted pentagonal ring and at least one substituted or unsubstituted naphthylene group; a fused ring of at least one substituted or unsubstituted pentagonal ring and at least one substituted or unsubstituted anthracenylene group a fused ring of at least one substituted or unsubstituted pentagonal ring and at least one substituted or unsubstituted phenanthrenylene group; or a combination thereof.

19. An electronic device comprising the stretchable polymer thin film of claim 12.

20. The electronic device of claim 19, wherein the electronic device includes at least one of an organic photoelectric device, an organic light emitting device, an organic diode, an organic thin film transistor, an organic solar cell, or an attachable device.
